(19) United States
(12) Patent Application Publication
(10) Pub. No.: US 2024/0279707 A1
(43) Pub. Date: Aug. 22, 2024

(54) PYRUVATE SENSOR AND RELATED METHOD

(71) Applicant: ABBOTT DIABETES CARE INC., Alameda, CA (US)

(72) Inventors: Tianmei Ouyang, Saratoga, CA (US); Stephen M. Oja, Reno, NV (US); Benjamin J. Feldman, Berkeley, CA (US)

(73) Assignee: ABBOTT DIABETES CARE INC., Alameda, CA (US)

(21) Appl. No.: 18/440,379

(22) Filed: Feb. 13, 2024

Related U.S. Application Data
(60) Provisional application No. 63/484,549, filed on Feb. 13, 2023.

(51) Int. Cl. C12Q 1/00; A61B 5/1486; C12Q 1/32

(52) U.S. Cl. CPC C12Q 1/005; A61B 5/14865; C12Q 1/32; C12Y 102/03003; G01N 2333/90203

(57) ABSTRACT

The present disclosure relates to sensing composition comprising a pyruvate-responsive enzyme and thiamine pyrophosphate (TPP). In particular, the sensing composition can comprise a pyruvate-responsive enzyme (e.g., pyruvate oxidase), TPP, optionally flavin adenine dinucleotide (FAD), a stabilizing agent (e.g., an albumin), and a redox mediator. The sensing composition forms a sensing layer on a working electrode to form a sensing electrode. The sensing electrode, along with a circuit, can form part of a system for sensing pyruvate. The present disclosure is further related to a method for sensing pyruvate, which includes accumulating charge derived from pyruvate reacting with the sensing composition for a set period of time and then measuring a signal from the accumulated charge.

### BACKGROUND

Detecting various analytes within an individual can be vital for monitoring the individual's health and well-being. Deviation from normal analyte levels can often be indicative of an underlying physiological condition, such as a metabolic condition or illness or exposure to particular environmental factors or stimuli. Glucose levels, for example, can be particularly important to detect and monitor in diabetic individuals.

Pyruvate is critical for numerous aspects of eukaryotic and human metabolism. Pyruvate is the end product of glycolysis and is ultimately destined for transport into mitochondria as a master fuel input undergirding citric acid cycle carbon flux. Appropriate regulation of pyruvate flux is critical for maintaining cellular function in multiple contexts. Metabolic dysfunction of pyruvate due to excessive inhibition of pyruvate dehydrogenase (PDH) by pyruvate dehydrogenase kinase (PDK) presents in chronic, progressive diseases such as chronic obstructive pulmonary disease (COPD), obesity, diabetes, and aging. For example, in patients with type 2 diabetes, increased serum and intramuscular lipids increase PDK activity, which in turn, reduces PDH activity and pyruvate flux into the citric acid cycle. It is believed that restoring normal pyruvate metabolism may relieve major aspects of the metabolic pathology present in type 2 diabetes. Otherwise healthy subjects with high-lipid, low-carbohydrate diets can have impaired PDH activity via PDK upregulation, so an understanding of pyruvate levels can be important for monitoring patient health.

Thus, there is a need for a biosensor for sensing pyruvate to monitor the health of patients, including those with diabetes.

### BRIEF SUMMARY

Some previous approaches to sensing pyruvate have relied on amperometric detection of the catalytic turnover of pyruvate using pyruvate-responsive enzymes such as pyruvate oxidase. Such sensors have allowed the crude detection of elevated pyruvate concentrations. However, such pyruvate concentrations may be non-reflective of physiological samples. Changes in pyruvate concentrations arising from changes in underlying biochemical processes may be very small and may not be easily detected using such sensors. Accordingly, there is a need for improved sensors and associated methods that are capable of reliably detecting low (e.g. micromolar) pyruvate concentrations.

The present disclosure reveals that, surprisingly, the sensitivity of a pyruvate sensor can be improved by controlling the concentration of cofactors for the pyruvate-responsive enzyme. The disclosure also reveals electrochemical methods that, surprisingly, improve the sensitivity of pyruvate sensors, such as those disclosed herein, to low pyruvate concentrations, such as may be present in physiological samples.

The disclosed compositions, sensors and methods offer significant advantages compared to those known in the art. For example, improved sensitivity is possible, meaning that lower concentrations of pyruvate can be detected. Because sensitivity of the response when sensing pyruvate can be improved, sensor elements can be designed which are less invasive, more sensitive, more reliable, and/or more cost-effective compared to known sensors. Because lower concentrations of pyruvate can be detected, more accurate information about health and wellness conditions can be obtained and changes which may have previously gone undetected can be monitored. The use of electrochemical techniques provided herein may further improve the sensitivity of pyruvate sensing that can be achieved. Such techniques may furthermore allow for electronics which have improved properties (e.g. increased lifetime in practical use, such as when used for continuous or semi-continuous pyruvate monitoring, reduced complexity, reduced size, and/or more cost-efficient construction).

The present disclosure relates to a sensing composition for detecting pyruvate. In some embodiments, the sensing composition can be used as part of a sensing electrode in a sensor. In some embodiments, the sensing composition can be used to produce a sensor for sensing pyruvate. The disclosure therefore also relates to a sensor which comprises the sensing composition. The disclosure also relates to a sensor obtainable from (e.g., formed from) the sensing composition. Methods of using the sensing composition and/or sensor to sense pyruvate are also provided.

In one embodiment the sensing composition comprises a pyruvate-responsive enzyme and thiamine pyrophosphate (TPP). In one embodiment the TPP is a cofactor for the pyruvate-responsive enzyme. In some embodiments, the pyruvate-responsive enzyme comprises pyruvate oxidase.

In some embodiments, the sensing composition further comprises one or more additional cofactors for the pyruvate-responsive enzyme. In some embodiments, the one or more additional cofactors comprise flavin adenine dinucleotide (FAD).

In some embodiments, the sensing composition further comprises a stabilizing agent (e.g., an enzyme-stabilizing agent). In some embodiments, the stabilizing agent is or comprises a protein. In some embodiments, the stabilizing agent is or comprises an albumin. In some embodiments, the albumin is bovine serum albumin (BSA) or human serum albumin (HSA).

In some embodiments, the pyruvate-responsive enzyme (e.g., pyruvate oxidase) and TPP are present in the composition, for example, and in certain embodiments, in a weight ratio of about 500:1 to about 1:1, such as about 100:1 to about 1:1 or about 50:1 to about 1:1 or about 10:1 to about 1:1.

In some embodiments, the sensing composition can further comprise a pH buffer.

In some embodiments, the redox mediator can comprise a polymer and an electron transfer agent. The polymer can comprise poly(4-vinylpyridine), poly(1-vinylimidazole), poly(thiophene), poly(aniline), poly(pyrrole), or poly(acetylene). In other embodiments, the polymer can comprise a polymer or copolymer repeat unit that can comprise at least one (e.g., 1, 2, 3, 4, 5, or 6) pendant pyridinyl group, imidazolyl group, or both a pyridinyl and imidazolyl group. In some embodiments, the electron transfer agent can comprise a transition metal complex, such as a transition metal complex that can comprise osmium, ruthenium, iron, cobalt, vanadium, or a combination thereof. In an embodiment, the transition metal complex can be an osmium transition metal complex that can comprise one or more ligands, wherein at least one (e.g., 1, 2, 3, 4, 5, or 6) ligand comprises a nitrogen-containing heterocycle. In an embodiment, the redox mediator can comprise an osmium complex bonded to a poly(4-vinylpyridine)-based polymer.

In some embodiments, the sensing composition can further comprise a cross linking agent. The cross linking agent can be a polyepoxide, cyanuric chloride, N-hydroxysuccinimide, an imidoester, epichlorohydrin, or a combination thereof. In an embodiment, the cross linking agent is a polyethylene glycol diglycidylether (PEGDGE).

In some embodiments, the sensing composition comprises an electron transfer agent. In some embodiments, the electron transfer agent is capable of transferring electrons from the pyruvate-responsive enzyme to a substrate such as an organic or inorganic surface, such as an electrode surface. In some embodiments, the electron transfer agent is a redox mediator. In some embodiments, the redox mediator is a redox polymer. In an embodiment, the pyruvate-responsive enzyme (e.g., pyruvate oxidase) can be immobilized (e.g., attached) to the redox mediator in the sensing composition. In some embodiments, the redox polymer is attached to the pyruvate-responsive enzyme. In some embodiments, the redox polymer is attached to the pyruvate-responsive enzyme and to a substrate such as an electrode surface.

In another embodiment, the present disclosure provides a sensing electrode that comprises a working electrode that can comprise a pyruvate sensing layer formed from the sensing composition described herein. The pyruvate sensing layer can be continuous or discontinuous. In an embodiment, the sensing electrode can further comprise a membrane overcoating at least the pyruvate sensing layer. In some embodiments, the membrane is permeable to pyruvate and/or the membrane has reduced permeability to TPP relative to pyruvate. In some embodiments, the membrane can comprise poly(4-vinylpyridine).

In another embodiment, the present disclosure provides a system for sensing pyruvate comprising a working electrode, a sensing element disposed on at least a portion of the working electrode, and a circuit configured to connect and disconnect with the working electrode, wherein the sensing element is configured to accumulate charge derived from pyruvate reacting with the pyruvate-responsive enzyme (e.g., pyruvate oxidase) for a set period of time. The sensing element is formed from the sensing composition as described herein. In an embodiment, the system can further comprise a sensor tail configured for insertion into a tissue, wherein the working electrode is disposed on the sensor tail.

The present disclosure also provides a method for sensing pyruvate comprising providing a sensing electrode as described herein, connecting the sensing electrode to a circuit to provide a steady state current, disconnecting the sensing electrode from the circuit, contacting the sensing electrode to an analyte that can comprise pyruvate, accumulating charge derived from pyruvate reacting with the pyruvate-responsive enzyme (e.g., pyruvate oxidase) and the redox mediator for a set period of time, connecting the sensing electrode to the circuit after the set period of time, and measuring a signal from the accumulated charge. In some embodiments, the set period of time is 30 seconds or more.

Additional embodiments and advantages of the disclosure will be set forth, in part, in the description that follows, and will flow from the description, or can be learned by practice of the disclosure.

It is to be understood that both the foregoing summary and the following detailed description are exemplary and explanatory only, and do not restrict the scope of the claims.

### BRIEF DESCRIPTION OF THE DRAWINGS/FIGURES

FIG. 1 is a schematic illustrating the sensor chemistry of pyruvate reacting with pyruvate oxidase in the presence of cofactor thiamine pyrophosphate (TPP), and an osmium-based redox material known as “X7,” which is attached (“wired”) to a working electrode.

FIG. 2 is a graph of current (nA) versus time (hours) over various pyruvate concentration that were tested against a sensing electrode prepared from a sensing composition comprising a pyruvate oxidase to TPP weight ratio of 200:1 (“1×TPP”).

FIG. 3 is a graph of current (nA) versus time (hours) over various pyruvate concentration that were tested against a sensing electrode prepared from a sensing composition comprising a pyruvate oxidase to TPP weight ratio of 1.73:1 (“100×TPP”).

FIG. 4 is a graph of accumulated charge (nC) versus pyruvate concentration (μM) for both the 1×TPP sensor (solid line) and 100×TPP sensor (dotted line).

### DETAILED DESCRIPTION

The headings provided herein are not limitations of the various embodiments of the disclosure, which can be defined by reference to the specification as a whole. It is also to be understood that the terminology used herein is for the purpose of describing particular embodiments only, and is not intended to be limiting, since the scope of the present disclosure will be limited only by the appended claims.

### Definitions

For convenience, the meaning of some terms and phrases used in the specification, examples, and appended claims are provided below. Unless stated otherwise, or implicit from context, the following terms and phrases include the meanings provided below. The definitions are provided to aid in describing particular embodiments, and are not intended to limit the claimed technology, because the scope of the technology is limited only by the claims. Unless otherwise defined, all technical and scientific terms used herein have the same meaning as commonly understood by one of ordinary skill in the art to which this technology belongs. If there is an apparent discrepancy between the usage of a term in the art and its definition provided herein, the definition provided within the specification will control.

The articles “a,” “an,” and “the” are used herein to refer to one or to more than one (i.e., to at least one) of the grammatical object of the article. By way of example, “an element” means one element or more than one element.

As used herein, the term “about” means±10% of the specified value, unless otherwise indicated.

The term “at least” prior to a number or series of numbers is understood to include the number associated with the term “at least,” and all subsequent numbers or integers that could logically be included, as clear from context. When at least is present before a series of numbers or a range, it is understood that “at least” can modify each of the numbers in the series or range. For example, “at least 3” means at least 3, at least 4, at least 5, etc. When at least is present before a component in a method step, then that component is included in the step, whereas additional components are optional.

As used herein, the terms “comprises,” “comprising,” “having,” “including,” “containing,” and the like are open-ended terms meaning “including, but not limited to.” To the extent a given embodiment disclosed herein “comprises” certain elements, it should be understood that present disclosure also specifically contemplates and discloses embodiments that “consist essentially of” those elements and that “consist of” those elements.

As used herein the terms “consists essentially of,” “consisting essentially of,” and the like are to be construed as a semi-closed terms, meaning that no other ingredients which materially affect the basic and novel characteristics of an embodiment are included.

As used herein, the terms “consists of,” “consisting of,” and the like are to be construed as closed terms, such that an embodiment “consisting of” a particular set of elements excludes any element, step, or ingredient not specified in the embodiment.

As used herein, the terms “measure,” “measuring,” and “measured” can encompass the meaning of a respective one or more of the terms “determine,” “determining,” “determined,” “calculate,” “calculating,” and “calculated.”

As used herein, an “analyte” is an enzyme substrate that is subject to be measured or detected. The analyte can be from, for example, a biofluid and can be tested in vivo, ex vivo, or in vitro. Pyruvate is an exemplary analyte in the present disclosure.

As used herein, a “sensor” is a device configured to detect the presence and/or absence and/or measure the level (e.g., concentration) of an analyte in a sample via electrochemical oxidation and reduction reactions on the sensor. These reactions are transduced to an electrical signal that can be correlated to an amount, concentration, or level of an analyte in the sample.

As used herein, a “working electrode” is an electrode at which the analyte (or a second compound whose level depends on the level of the analyte) is electrooxidized or electroreduced with or without the agency of an electron transfer agent.

As used herein, a “counter electrode” refers to an electrode paired with the working electrode, through which passes a current equal in magnitude and opposite in sign to the current passing through the working electrode. In the context of embodiments of the present disclosure, the term “counter electrode” includes both a) counter electrodes and b) counter electrodes that also function as reference electrodes (i.e., counter/reference electrodes), unless otherwise indicated.

As used herein, a “reference electrode” includes both a) reference electrodes and b) reference electrodes that also function as counter electrodes (i.e., counter/reference electrodes), unless otherwise indicated.

As used herein, “electrolysis” is the electrooxidation or electroreduction of a compound either directly at an electrode or via one or more electron transfer agents.

As used herein, components are “immobilized” or “attached” to a polymer and/or a sensor, for example, when the components are entrapped on, entrapped within, covalently bound, ionically bound, electrostatically bound, or coordinatively bound to constituents of a polymer, a sol-gel matric, membrane, and/or sensor, which reduces or precludes mobility.

As used herein, a “non-leachable,” or “non-releasable” compound, or a compound that is “non-leachably disposed” is meant to define a compound that is affixed on the sensor such that it does not substantially diffuse away from the sensing layer of the working electrode for the period in which the sensor is used (e.g., the period in which the sensor is implanted in a patient or measuring a sample).

As used herein an “electron transfer agent” is a compound that carries electrons between the analyte and the working electrode, either directly, or in cooperation with other electron transfer agents. One example of an electron transfer agent is a redox mediator.

As used herein, a “redox mediator” is an electron-transfer agent for carrying electrons between an analyte, an analyte-reduced or analyte-oxidized, enzyme, and an electrode, either directly, or via one or more additional electron-transfer agents. A redox mediator that includes a polymeric backbone can also be referred to as a “redox polymer.”

As used herein, the term “precursor polymer” refers to the starting polymer before the various modifier groups are attached to form a modified polymer.

A “substituted” functional group (e.g., substituted alkyl, alkenyl, alkoxy, aryl) includes at least one substituent (e.g., 1, 2, 3, 4, or 5) that can be, for example, halo, alkoxy, mercapto, aryl, alkoxycarbonyl, alkylaminocarbonyl, dialkylaminocarbonyl, hydroxy, amino, alkylamino, dialkylamino, trialkylammonium, alkanoylamino, arylcarboxamido, hydrazino, alkylthio, alkenyl, and reactive groups.

A “reactive group” is a functional group of a molecule (e.g., a polymer, a crosslinking agent, an enzyme) that is capable of reacting with another compound to couple at least a portion (e.g., another reactive group) of that other compound to the molecule. Reactive groups include carboxy, activated ester, sulfonyl halide, sulfonate ester, isocyanate, isothiocyanate, epoxide, aziridine, halide, aldehyde, ketone, amine, acrylamide, thiol, acyl azide, acyl halide, hydrazine, hydroxylamine, alkyl halide, imidazole, pyridine, phenol, alkyl sulfonate, halotriazine, imido ester, maleimide, hydrazide, hydroxy, and photo-reactive azido aryl groups. Activated esters, as understood in the art, generally include esters of succinimidyl, benzotriazolyl, or aryl substituted by electron-withdrawing groups such as sulfo, nitro, cyano, or halo groups; or carboxylic acids activated by carbodiimides.

As used herein, a “sensing layer” is a component of the sensor including constituents that facilitate the electrolysis of the analyte. The sensing layer can include constituents such as a redox mediator (e.g., an electron transfer agent or a redox polymer), a catalyst (e.g., an analyte-specific enzyme), which catalyzes a reaction of the analyte to produce a response at the working electrode, or both an electron transfer agent and a catalyst. In some embodiments of the present disclosure, a sensor includes a sensing layer that is non-leachably disposed in proximity to or on the working electrode.

As used herein, a “sensing element” is an application or region of an analyte-specific enzyme disposed with the sensing layer. As such, a sensing element is capable of interacting with the analyte. A sensing layer can have more than one sensing element making up the analyte detection area disposed on the working electrode. In some embodiments, the sensing element includes an analyte-specific enzyme and an electron transfer agent (e.g., electron transfer agent). In some embodiments, the sensing element includes an analyte specific enzyme, a redox mediator, and a crosslinker.

As used herein, “crosslinking agent” or “crosslinker” is a molecule that contains at least two (e.g., 2, 3, or 4) reactive groups (e.g., terminal functional groups) that can link at least two molecules together (intermolecular crosslinking) or at least two portions of the same molecule together (intramolecular crosslinking). A crosslinking agent having more than two reactive groups can be capable of both intermolecular and intramolecular crosslinkings at the same time.

A “membrane solution” is a solution that contains the components for crosslinking and forming the membrane, including, e.g., polymer (e.g., a modified polymer containing heterocyclic nitrogen groups), a crosslinking agent, and a solvent (e.g., a buffer or an alcohol-buffer mixed solvent).

As used herein, “configured to accumulate charge” is an arrangement of the working electrode and circuit that allows for the accumulation of electrons produced from the oxidation of the analyte (e.g., pyruvate). The oxidation occurring at or on the sensing element of the working electrode is not connected to the circuit, thereby creating the accumulation of electrons.

As used herein, “a set period of time” is the amount of time needed to accumulate the charge (electrons) to provide a sufficient signal output that can be measured and quantified for a given analyte (e.g., pyruvate). Typically, and as discussed in detail elsewhere herein, the set period of time is 30 seconds or more and 30 minutes or less.

As used herein, a “biofluid” is any bodily fluid or bodily fluid derivative in which the analyte can be measured. Examples of biofluid include, for example, dermal fluid, subcutaneous fluid, interstitial fluid, plasma, blood (e.g., from a vein or blood vessel), lymph, synovial fluid, cerebrospinal fluid, saliva, bronchoalveolar lavage, amniotic fluid, sweat, or tears.

The term “patient” refers to a living animal, and thus encompasses a living mammal and a living human, for example. The term “user” can be used herein as a term that encompasses the term “patient.”

As used herein, “accumulation mode sensing” refers to the accumulation of electrons produced from the oxidation of an analyte, the oxidation occurring at or on the sensing element of a working electrode that is not connected to a circuit, thereby creating the accumulation of electrons.

### Compositions and Methods of the Disclosure

In an embodiment, the present disclosure is directed to a sensing composition for detecting pyruvate. In some embodiments, the sensing composition comprises a pyruvate-responsive enzyme and thiamine pyrophosphate (TPP). In some embodiments, the pyruvate-responsive enzyme is pyruvate oxidase.

In some embodiments, the sensing composition comprises further components. In some embodiments, the sensing composition comprises one or more cofactors of the pyruvate-responsive enzyme (e.g. FAD); and/or one or more enzyme stabilizing agents (e.g., an albumin). In some embodiments, the sensing composition comprises an electron-transfer agent, such as a redox mediator.

In an embodiment, the present disclosure is directed to a sensing composition comprising a pyruvate-responsive enzyme (e.g., pyruvate oxidase), thiamine pyrophosphate (TPP), flavin adenine dinucleotide (FAD), an albumin, and a redox mediator.

In some embodiments, the composition comprises from about 1 to about 100 mg/mL of the pyruvate-responsive enzyme (e.g., pyruvate oxidase), such as from about 5 to about 50 mg/mL, e.g., from about 10 to about 30 mg/mL or about 20 mg/mL.

In some embodiments, the composition comprises from about 0.04 mg/mL to about 40 mg/mL TPP, such as from about 0.1 to about 20 mg/mL, such as from about 0.5 to about 18 mg/mL, e.g, from about 1 mg/mL to about 16 mg/mL, such as from about 5 mg/mL to about 15 mg/mL, e.g., from about 10 to about 12 mg/mL TPP.

In some embodiments, the relative amounts of the pyruvate-responsive enzyme and the TPP are expressed in terms of a weight ratio of the pyruvate-responsive enzyme (e.g., pyruvate oxidase) to the TPP. In some embodiments, the pyruvate-responsive enzyme (e.g., pyruvate oxidase) and TPP are present in the composition in a weight ratio of about 500:1 to about 1:1. For example, the TPP is used in a concentration that ranges from 1/500th of the concentration of the pyruvate-responsive enzyme (e.g., pyruvate oxidase) to an equal concentration of the pyruvate-responsive enzyme (e.g., pyruvate oxidase). The TPP and FAD are cofactors to a pyruvate-responsive enzyme (e.g., pyruvate oxidase). It was unexpectedly discovered that the amount of TPP in the composition affects the ability of a sensor to sufficiently detect pyruvate. It was further discovered that by increasing the ratio of TPP to pyruvate-responsive enzyme the sensitivity of pyruvate sensing using the composition can be increased. Advantageously, a minimum concentration of TPP within the sensing composition can be used to affect detection. In particular, it has been surprisingly discovered that a quantity of TPP that is about one five hundredth or more the amount of a pyruvate-responsive enzyme (e.g., pyruvate oxidase) by weight should be used in the composition and its use in a sensing method for pyruvate to be detected. Accordingly, in some embodiments, the pyruvate-responsive enzyme (e.g., pyruvate oxidase) and TPP are present in the sensing composition described herein such that the weight ratio of the pyruvate-responsive enzyme (e.g., pyruvate oxidase) to TPP is at least 500:1. In some embodiments, the weight ratio of the pyruvate-responsive enzyme (e.g., pyruvate oxidase) to TPP can be at least about 500:1, at least about 400:1, at least about 300:1, at least about 200:1, at least about 100:1, at least about 50:1, at least about 20:1, at least about 10:1, at least about 5:1, at least about 4:1, at least about 3:1, or at least about 2:1.

In some embodiments, the weight ratio of the pyruvate-responsive enzyme (e.g., pyruvate oxidase) to TPP can be in a range of about 500:1 to about 1:1 (e.g., about 450:1 to about 1:1, about 400:1 to about 1:1, about 350:1 to about 1:1, about 300:1 to about 1:1, about 250:1 to about 1:1, about 200:1 to about 1:1, about 150:1 to about 1:1, about 100:1 to about 1:1, about 90:1 to about 1:1, about 80:1 to about 1:1, about 70:1 to about 1:1, about 60:1 to about 1:1, about 50:1 to about 1:1, about 45:1 to about 1:1, about 40 to about 1:1, about 35:1 to about 1:1, about 30:1 to about 1:1, about 25:1 to about 1:1, about 20:1 to about 1:1, about 15:1 to about 1:1, about 10:1 to about 1:1, about 9:1 to about 1:1, about 8:1 to about 1:1, about 7:1 to about 1:1, about 6:1 to about 1:1, about 5:1 to about 1:1, about 4:1 to about 1:1, about 3:1 to about 1:1, or about 2:1 to about 1:1). For example, the weight ratio of the pyruvate-responsive enzyme (e.g., pyruvate oxidase) to TPP can be about 500:1, about 475:1, about 450:1, about 425:1, about 400:1, about 375:1, about 350:1, about 325:1, about 300:1, about 275:1, about 250:1, about 225:1, about 200:1, about 175:1, about 150:1, about 125:1, about 100:1, about 95:1, about 90:1, about 85:1, about 80:1, about 75:1, about 70:1, about 65:1, about 60:1, about 55:1, about 50:1, about 45:1, about 50:1, about 45:1, about 50:1, about 45:1, about 40:1, about 35:1, about 30:1, about 25:1, about 20:1, about 15:1, about 10:1, about 9.5:1, about 9:1, about 8.5:1, about 8:1, about 7.5:1, about 7:1, about 6.5:1, about 6:1, about 5.5:1, about 5:1, about 4:1, about 3.5:1, about 3:1, about 2.5:1, about 2:1, about 1.5:1, or about 1:1. In an embodiment, the weight ratio of the pyruvate-responsive enzyme (e.g., pyruvate oxidase) to TPP can be less than about 200:1 to about 1:1. In an embodiment, the weight ratio of the pyruvate-responsive enzyme (e.g., pyruvate oxidase) to TPP can be about 150:1 to about 1:1. In an embodiment, the weight ratio of the pyruvate-responsive enzyme (e.g., pyruvate oxidase) to TPP can be about 100:1 to about 1:1. In an embodiment, the weight ratio of the pyruvate-responsive enzyme (e.g., pyruvate oxidase) to TPP can be about 50:1 to about 1:1. In an embodiment, the weight ratio of the pyruvate-responsive enzyme (e.g., pyruvate oxidase) to TPP can be about 25:1 to about 1:1. In an embodiment, the weight ratio of the pyruvate-responsive enzyme (e.g., pyruvate oxidase) to TPP can be about 10:1 to about 1:1. In an embodiment, the weight ratio of the pyruvate-responsive enzyme (e.g., pyruvate oxidase) to TPP can be about 5:1 to about 1:1. In an embodiment, the weight ratio of the pyruvate-responsive enzyme (e.g., pyruvate oxidase) to TPP can be about 1:1 to about 3:1 or about 1:1 to about 2.5:1, or about 2:1 to about 1:1. In certain embodiments, the weight ratio of the pyruvate-responsive enzyme (e.g., pyruvate oxidase) to TPP can be about 2:1.

Those skilled in the art will appreciate that the weight ratio of the pyruvate-responsive enzyme (e.g., pyruvate oxidase) to TPP can be calculated based on, for example, the concentrations of the agents in a composition. By way of non-limiting illustration, a composition comprising 20 mg/mL of pyruvate-responsive enzyme (e.g., pyruvate oxidase) and 0.1 mg/mL has a weight ratio of pyruvate-responsive enzyme (e.g., pyruvate oxidase) to TPP of 200:1; and a composition comprising 20 mg/mL of pyruvate-responsive enzyme (e.g., pyruvate oxidase) and 11.5 mg/mL has a weight ratio of pyruvate-responsive enzyme (e.g., pyruvate oxidase) to TPP of 1.7:1 (to one decimal place).

The amounts of the pyruvate-responsive enzyme (e.g., pyruvate oxidase) and TPP present in the sensing composition described can also be expressed in terms of weight ratio of the TPP to pyruvate-responsive enzyme (e.g., pyruvate oxidase). A weight ratio of pyruvate-responsive enzyme (e.g., pyruvate oxidase) to TPP of 500:1 corresponds to a weight ratio of the TPP to pyruvate-responsive enzyme (e.g., pyruvate oxidase) of 0.002:1. Thus, a composition comprising 20 mg/mL of pyruvate-responsive enzyme (e.g., pyruvate oxidase) and 0.1 mg/mL has a weight ratio of TPP to pyruvate-responsive enzyme (e.g., pyruvate oxidase) of 0.005:1; and a composition comprising 20 mg/mL of pyruvate-responsive enzyme (e.g., pyruvate oxidase) and 11.5 mg/mL has a weight ratio of TPP to pyruvate-responsive enzyme (e.g., pyruvate oxidase) of 0.575:1.

In some embodiments, therefore, the weight ratio of TPP to pyruvate-responsive enzyme (e.g., pyruvate oxidase) is at least 0.002:1. In some embodiments, the weight ratio of TPP to pyruvate-responsive enzyme (e.g., pyruvate oxidase) is at least about 0.005:1, e.g. at least about 0.01:1, e.g., about 0.02:1, e.g. at least about 0.05:1, e.g., at least about 0.1:1, e.g., at least about 0.2:1, e.g., at least about 0.25:1, e.g., at least about 0.33:1, e.g., at least about 0.5:1.

In some embodiments, the weight ratio of TPP to pyruvate-responsive enzyme (e.g., pyruvate oxidase) is in a range of from about 0.002:1 to about 1:1, such as from about 0.005:1 to 1:1, e.g., about 0.01:1 to 1:1, e.g., from 0.02:1 to 1:1, e.g., about 0.05:1 to 1:1, e.g., about 0.1:1 to 1:1, e.g., about 0.2:1 to 1:1, e.g., about 0.25:1 to 1:1, e.g., about 0.33:1 to 1:1, e.g., about 0.5:1 to 1:1.

In some embodiments, the relative amounts of the pyruvate-responsive enzyme and the TPP are expressed in terms of a molar ratio of the TPP to the pyruvate-responsive enzyme (e.g. pyruvate oxidase).

In some embodiments, the TPP is in molar excess relative to the pyruvate-responsive enzyme (e.g., pyruvate oxidase). In some embodiments, the molar ratio of TPP to the pyruvate-responsive enzyme (e.g., pyruvate oxidase) can be at least about 1:1, at least about 2:1, at least about 3:1, at least about 4:1, at least about 5:1, at least about 10:1, at least about 20:1, at least about 50:1, at least about 100:1, at least about 200:1, at least about 300:1, or more.

In some embodiments, the molar ratio is from about 1.2:1 to about 611.3:1, e.g., from about 1.3:1 to about 611.3:1, e.g., from about 1.4:1 to about 611.3:1, e.g., from about 1.4:1 to about 611.3:1, e.g., from about 1.5:1 to about 611.3:1, e.g., from about 1.6:1 to about 611.3:1, e.g., from about 1.7:1 to about 611.3:1, e.g., from about 1.9:1 to about 611.3:1, e.g., from about 2.0:1 to about 611.3:1, e.g., from about 2.2:1 to about 611.3:1, e.g., from about 2.4:1 to about 611.3:1, e.g., from about 2.7:1 to about 611.3:1, e.g., from about 3.1:1 to about 611.3:1, e.g., from about 3.5:1 to about 611.3:1, e.g., from about 4.1:1 to about 611.3:1, e.g., from about 4.9:1 to about 611.3:1, e.g., from about 6.1:1 to about 611.3:1, e.g., from about 6.8:1 to about 611.3:1, e.g., from about 7.6:1 to about 611.3:1, e.g., from about 8.7:1 to about 611.3:1, e.g., from about 10.2:1 to about 611.3:1, e.g., from about 12.2:1 to about 611.3:1, e.g., from about 13.6:1 to about 611.3:1, e.g., from about 15.3:1 to about 611.3:1, e.g., from about 17.5:1 to about 611.3:1, e.g., from about 20.4:1 to about 611.3:1, e.g., from about 24.5:1 to about 611.3:1, e.g., from about 30.6:1 to about 611.3:1, e.g., from about 40.8:1 to about 611.3:1, e.g., from about 61.1:1 to about 611.3:1, e.g., from about 67.9:1 to about 611.3:1, e.g., from about 76.4:1 to about 611.3:1, e.g., from about 87.3:1 to about 611.3:1, e.g., from about 101.9:1 to about 611.3:1, e.g., from about 122.3:1 to about 611.3:1, e.g., from about 152.8:1 to about 611.3:1, e.g., from about 174.7:1 to about 611.3:1, e.g., from about 203.8:1 to about 611.3:1, e.g., from about 244.5:1 to about 611.3:1, e.g., from about 305.7:1 to about 611.3:1.

The pyruvate-responsive enzyme is any enzyme or combination of enzymes that converts pyruvate (or a pyruvate-TPP adduct) into acetylphosphate. In some embodiments, the pyruvate-responsive enzyme can comprise pyruvate oxidase, pyruvate dehydrogenase, pyruvate decarboxylase, or a combination thereof. In some embodiments, the pyruvate-responsive enzyme is pyruvate oxidase. In some embodiments, one or more cofactors (e.g., coenzymes) can be used in combination with the pyruvate-responsive enzyme. In some embodiments, the cofactor can be thiamine pyrophosphate (TPP), flavin adenine dinucleotide (FAD), or a combination thereof. In some embodiments, TPP is a cofactor for the pyruvate-responsive enzyme. In some embodiments, the pyruvate-responsive enzyme is a TPP-dependent enzyme. In some embodiments, the pyruvate-responsive enzyme is pyruvate oxidase. In some embodiments, the pyruvate-responsive enzyme is a phosphate-dependent pyruvate oxidase.

When the pyruvate-responsive enzyme is or comprises pyruvate oxidase, any suitable pyruvate oxidase enzyme can be used. Pyruvate oxidase enzymes are commercially available from suppliers, such as Toyobo USA (New York). In some embodiments, the pyruvate oxidase is derived from Pseudomonas sp. In some embodiments, the pyruvate oxidase has an Enzyme Commission number of EC 1.2.3.3 and/or a Chemical Abstracts Service (CAS) Registry Number of 9001-96-1. In some embodiments, the pyruvate oxidase is an enzyme

Typically the pyruvate-responsive enzyme has a molecular weight of from about 100 to about 400 kDa, such as from about 200 to about 300 kDa, e.g. from about 220 to about 280 kDa such as from about 250 to about 270 kDa e.g., from 260 kDa. An exemplary pyruvate oxidase is commercially available as product PYO-311 from Toyoba USA and has a molecular weight of about 260 kDa.

The sensing composition can further comprise a stabilizing agent (e.g., an enzyme-stabilizing agent). In some embodiments, the stabilizing agent is or comprises a protein, such as an albumin or casein, catalase, or is a small organic molecule, such as a polyol, a carboxylic acid, a carboxylic acid salt, a carboxylic acid ester, and a sugar, or any combination thereof. In some embodiments, the stabilizing agent is an albumin. In an embodiment, the albumin can be a serum albumin, such as bovine serum albumin or human serum albumin. In certain embodiments, the sensing composition can further comprise bovine serum albumin.

In some embodiments, the albumin is present in the composition in an amount of from about 1 to about 50 mg/mL, such as from about 2 to about 20 mg/mL, such as from about 5 to about 15 mg/mL, e.g., about 10 mg/mL.

In some embodiments, the sensing composition comprises a pyruvate-responsive enzyme, thiamine pyrophosphate (TPP), flavin adenine dinucleotide (FAD), an albumin, and a redox mediator.

In some embodiments, the sensing composition comprises FAD. In some embodiments, the FAD is present in the composition in an amount of from about 0.01 to about 1 mg/mL, such as from about 0.05 to about 0.5 mg/mL, e.g., about 0.1 mg/mL.

In any of the embodiments, the sensing composition can comprise a pH buffer. The buffer can be any suitable composition that is water soluble and controls (i.e., maintains) the pH of the sensing composition within a pH of about 5 to about 8 (e.g., maintains a pH of about 5, about 5.5, about 6, about 6.5, about 7, about 7.5, or about 8). In some embodiments, the pH can be controlled to be within a range of about 6 to about 8. For example, the buffer can comprise a phosphate (e.g., monobasic and dibasic sodium phosphate), 4-(2-hydroxyethyl)piperazine-1-ethanesulfonic acid (HEPES), 3-(N-morpholino) propanesulfonic acid (MOPS), 2-amino-2-(hydroxymethyl)-1,3-propanediol (TRIS), a carbonate (e.g., carbonic acid and a carbonate salt, such as sodium carbonate; sodium carbonate and sodium bicarbonate), or a citrate (e.g., citric acid and a citrate salt, such as trisodium citrate). The buffer can optionally comprise one or more (e.g., 1, 2, 3, or 4) additional salts (e.g., Group I or Group II halide salts, e.g., sodium chloride, potassium chloride, magnesium chloride). In an embodiment, the buffer can be phosphate-buffered saline (PBS), which comprises disodium hydrogen phosphate, sodium chloride, and optionally potassium chloride and potassium dihydrogen phosphate. In another embodiment, the buffer can be HEPES or a phosphate buffer that can comprise phosphate, sodium chloride, and magnesium chloride. In some embodiments, the sensing composition comprises buffer salts at a concentration of from about 10 mM to about 500 mM, such as from about 20 mM to about 100 mM, e.g., from 50 mM. For example, in some embodiments, the sensing composition comprises about 10 mM to about 500 mM, such as from about 20 mM to about 100 mM, e.g., from 50 mM phosphate (e.g. sodium phosphate). In some embodiments, the sensing composition comprises one or more Group I or II halide salts at a concentration of from about 10 mM to about 500 mM, such as from about 20 mM to about 100 mM, e.g., from 50 mM to about 60 mM. In some embodiments, the sensing composition comprises about 10 mM to about 500 mM, such as from about 20 mM to about 100 mM, e.g., from 50 mM to about 60 mM NaCl and/or MgCl₂, such as about 50 mM NaCl and about 10 mM MgCl₂. In some embodiments, the sensing composition comprises about 50 mM phosphate, about 50 mM NaCl, and about 10 mM MgCl₂, and is buffered to about pH 6.

The buffer typically is an aqueous buffer but other non-aqueous solvents can be present, such as an alcohol (e.g., ethanol). In some embodiments, the buffer comprises water as the only solvent. In other embodiment, the buffer can comprise water and at least one (e.g., 1, 2, or 3) non-aqueous solvents in any suitable ratio, such as a non-aqueous solvent to water volume ratio ranging from 99.9:0.1 to 0.1:99.9. In some embodiments, the non-aqueous solvent to water volume ratio is about 1:99, about 5:95, about 10:90, about 15:85, about 20:80, about 25:75, about 30:70, about 35:65, about 40:60, about 45:55, about 50:50, about 55:45, about 60:40, about 65:35, about 70:30, about 75:25, about 80:20, about 85:15, about 90:10, about 95:5, or about 99:1, etc.). In a specific example, ethanol (EtOH) and water are used in a volume ratio ranging from 50:50 to 90:10 EtOH:H₂O (e.g., 70:30, about 75:25, about 80:20, about 85:15, or about 90:10, etc.).

In an embodiment, the redox mediator can comprise a polymer and an electron transfer agent.

The polymer in the redox mediator can be any suitable polymer that allows the transfer of electrons between the electron transfer agent and the working electrode. For example, the polymer can be, poly(4-vinylpyridine), poly(1-vinylimidazole), poly(thiophene), poly(aniline), poly(pyrrole), poly(acetylene), poly(acrylic acid), styrene/maleic anhydride copolymer, methylvinylether/maleic anhydride copolymer, poly(vinylbenzylchloride), poly(allylamine), poly(lysine), poly(acrylamide-co-1-vinyl imidazole), poly(4-vinylpyridine) quaternized with carboxypentyl groups, and poly(sodium 4-styrene sulfonate). These polymers can be considered precursor polymers in that the polymers are further modified to immobilize (e.g., attach) the electron transfer complex. In some embodiments, the polymer can comprise poly(4-vinylpyridine), poly(1-vinylimidazole), poly(thiophene), poly(aniline), poly(pyrrole), or poly(acetylene). In other embodiments, the polymer can comprise a polymer or copolymer repeat unit that can comprise at least one (e.g., 1, 2, 3, 4, 5, or 6) pendant pyridinyl group, imidazolyl group, or both a pyridinyl and imidazolyl group. For example, a suitable polymer includes partially or fully quaternized poly(4-vinylpyridine) and poly(1-vinylimidazole) in which quaternized pyridine and imidazole groups, respectively, can be used to form spacers by reaction with (e.g., complexation with) an electron transfer agent.

The electron transfer agent in the redox mediator typically comprises a transition metal complex. The transition metal in the transition metal complex can be any suitable transition metal that can be effectively reduced and oxidized in the method described herein. For example, the transition metal complex can comprise osmium, ruthenium, iron, cobalt, vanadium, or a combination thereof. In some embodiments, the transition metal can be ruthenium or osmium, particularly osmium. According to some embodiments, suitable electron transfer agents can include low-potential osmium complexes, such as those described in U.S. Pat. Nos. 6,134,461, 6,605,200, 6,736,957, 7,501,053, and 7,754,093, the disclosures of each of which are incorporated herein by reference in their entirety. Other suitable examples of electron transfer mediators and polymer-bound electron transfer mediators can include those described in U.S. Pat. Nos. 8,444,834, 8,268,143, and 6,605,201, the disclosures of which are incorporated herein by reference in their entirety.

The transition metal complex can further comprise at least one ligand, which can be monodentate or multidentate (e.g., bidentate, tridentate, tetradentate). Typically the complex will include enough ligands to provide a full coordination sphere. In some embodiments, at least one ligand (e.g., 1, 2, 3, 4, 5, or 6) can comprise a nitrogen-containing heterocycle.

Monodentate ligands include, for example, —F, —Cl, —Br, —I, —CN, —SCN, —OH, NH₃, alkylamine, dialkylamine, trialkylamine, alkoxy, a heterocyclic compound, compounds containing such groups, a solvent molecule (e.g., H₂O, EtOH), or a reactive group. For example, an alkyl (e.g., C₁₋₁₂, C₁₋₆, C₁₋₄, C₁₋₃) or aryl (e.g., phenyl, benzyl, naphthyl) portions of a ligand can be optionally substituted by, e.g., F, Cl, Br, I, alkylamino, dialkylamino, trialkylammonium (except aryl portions), alkoxy, alkylthio, and aryl. Examples of suitable heterocyclic monodentate ligands include imidazole, pyrazole, oxazole, thiazole, pyridine, and pyrazine, each of which can be unsubstituted or substituted, as described herein (e.g., with at least one reactive group, such as 1, 2, 3, or 4 reactive groups).

Examples of suitable bidentate ligands include, for example, 1,10-phenanthroline, an amino acid, oxalic acid, acetylacetone, a diaminoalkane, an ortho-diaminoarene, 2,2′-biimidazole, 2,2′-bioxazole, 2,2′-bithiazole, 2-(2-pyridyl)imidazole, and 2,2′-bipyridine, each of which can be unsubstituted or substituted, as described herein (e.g., substituted with at least one reactive group, such as 1, 2, 3, or 4 reactive groups). Particularly suitable bidentate ligands for the electron transfer complex include substituted and unsubstituted 2,2′-biimidazole, 2-(2-pyridyl)imidazole, and 2,2′-bipyridine. Examples of suitable terdentate ligands include, for example, diethylenetriamine, 2,2′,2″-terpyridine, 2,6-bis(N-pyrazolyl)pyridine, each of which can substituted or unsubstituted (e.g., substituted with one more alkyl groups, such as methyl, or one or more reactive groups).

A suitable 2,2′-biimidazole ligand can be a ligand according to formula (I):

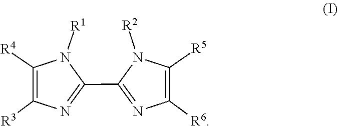

In formula (I), R¹ and R² are the same or different and each is a substituted or unsubstituted alkyl, alkenyl, or aryl. Generally, R¹ and R² are the same or different and each is an unsubstituted C₁₋₁₂ alkyl (e.g., C₁₋₄ alkyl). In some embodiments, both R¹ and R² are methyl.

In formula (I), R³, R⁴, R⁵, and R⁶ are the same or different and each is H, F, Cl, Br, I, NO₂, CN, CO₂H, SO₃H, SH, alkoxycarbonyl, alkylaminocarbonyl, dialkylaminocarbonyl, hydroxy, alkoxy, amino, alkylamino, dialkylamino, alkanoylamino, arylcarboxamido, hydrazino, alkylhydrazino, hydroxylamino, alkoxyamino, alkylthio, alkyl, alkenyl, or aryl. Alternatively, R³ and R⁴, in combination, or R⁵ and R⁶, in combination, independently form a saturated or unsaturated 5- or 6-membered ring (e.g., benzo). Typically, the alkyl and alkoxy portions are C₁₋₁₂. The alkyl or aryl portions of any of the substituents can be optionally substituted by one or more substituents (e.g., 1, 2, 3, 4, 5, or 6), such as F, Cl, Br, I, amino, alkylamino, dialkylamino, trialkylammonium (except on aryl portions), alkoxy, alkylthio, aryl, or a reactive group (e.g., CO₂H). Generally, R³, R⁴, R⁵, and R⁶ are the same or different and each is H or an unsubstituted C₁₋₁₂ alkyl (e.g., C₁₋₄ alkyl). In some embodiments, R³, R⁴, R⁵, and R⁶ are all H.

A suitable 2-(2-pyridyl)imidazole ligand can be a ligand according to formula (II):

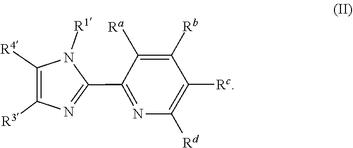

In formula (II), R is a substituted or unsubstituted alkyl, alkenyl, or aryl. Generally, R¹ is an unsubstituted C₁₋₁₂ alkyl (e.g., C₁₋₄ alkyl) or a C₁₋₁₂ alkyl that is optionally substituted with a reactive group. In some embodiments, R¹ is methyl.

In formula (II), R^{3′}, R^{4′}, R^{a}, R^{b}, R^{c}, and R^{d} are the same or different and each is H, F, Cl, Br, I, NO₂, CN, CO₂H, SO₃H, SH, alkoxycarbonyl, alkylaminocarbonyl, dialkylaminocarbonyl, hydroxy, alkoxy, amino, alkylamino, dialkylamino, alkanoylamino, arylcarboxamido, hydrazino, alkylhydrazino, hydroxylamino, alkoxyamino, alkylthio, alkyl, alkenyl, or aryl. Alternatively, R^{3′} and R^{4′}, in combination, or two adjacent substituents of R^{a}, R^{b}, R^{c}, and R^{d} (e.g., R^{a} and R^{b}, R^{b} and R^{c}, or R^{c} and R^{d}) in combination, independently form a saturated or unsaturated 5- or 6-membered ring (e.g., benzo). Typically, the alkyl and alkoxy portions are C₁₋₁₂. The alkyl or aryl portions of any of the substituents can be optionally substituted by one or more substituents (e.g., 1, 2, 3, 4, 5, or 6), such as F, Cl, Br, I, amino, alkylamino, dialkylamino, trialkylammonium (except on aryl portions), alkoxy, alkylthio, aryl, or a reactive group (e.g., CO₂H). Generally, R^{3′}, R^{4′}, R^{a}, R^{b}, R^{c}, and R^{d} are the same or different and each is H or an unsubstituted C₁₋₁₂ alkyl (e.g., C₁₋₄ alkyl). In some embodiments, R^{3′}, R^{4′}, R^{a}, R^{b}, R^{c}, and R^{d} are all H.

A suitable 2,2′-bipyridine ligand can be a ligand according to formula (III):

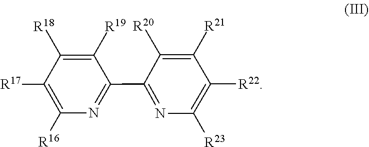

In formula (III), R¹⁶, R¹⁷, R¹⁸, R¹⁹, R²⁰, R²¹, R²², and R²³ are the same or different and each is H, F, Cl, Br, I, NO₂, CN, CO₂H, SO₃H, SH, alkoxycarbonyl, alkylaminocarbonyl, dialkylaminocarbonyl, hydroxy, alkoxy, amino, alkylamino, dialkylamino, alkanoylamino, arylcarboxamido, hydrazino, alkylhydrazino, hydroxylamino, alkoxyamino, alkylthio, alkyl, alkenyl, or aryl. Typically, the alkyl and alkoxy portions are C₁₋₁₂. The alkyl or aryl portions of any of the substituents can be optionally substituted by one or more substituents (e.g., 1, 2, 3, 4, 5, or 6), such as F, Cl, Br, I, amino, alkylamino, dialkylamino, trialkylammonium (except on aryl portions), alkoxy, alkylthio, aryl, or a reactive group (e.g., CO₂H).

Specific examples of suitable combinations include R¹⁶ and R²³ are both H or both methyl and/or R¹⁷ and R²³ are both H or both methyl and/or R¹⁸ and R²¹ are both H or both methyl and/or R¹⁹ and R²⁰ are both H or both methyl. An alternative combination is where one or more adjacent pairs of substituents (e.g., R¹⁶ and R¹⁷, R¹⁷ and R¹⁸, R¹⁸ and R¹⁹, R²³ and R²², R²² and R²¹, or R²¹ and R²⁰), in combination, form a saturated or unsaturated 5- or 6-membered ring (e.g., benzo).

In an embodiment, the one or more ligand is 4,4′-dimethyl-2,2′-bipyridine, mono-, di-, or polyalkoxy-2,2′-bipyridines (e.g., 4,4′-dimethoxy-2,2′-bipyridine), 4,7-dimethyl-1,10-phenanthroline, mono, di-, or polyalkoxy-1,10-phenanthrolines (e.g., 4,7-dimethoxy-1,10-phenanthroline), or a combination of any of these.

In some embodiments, the transition metal complex will include a counterion (X) to balance the charge of the transition metal. Typically, there will be 1 to 5 (i.e., 1, 2, 3, 4, or 5) counterions. Multiple counterions in the complex are not necessarily all the same. Examples of suitable counterions include anions, such as halide (e.g., fluoride, chloride, bromide, or iodide), sulfate, phosphate, hexafluorophosphate, and tetrafluoroborate, and cations (e.g., a monovalent cation), such as lithium, sodium, potassium, tetralkylammonium, and ammonium. In some embodiments, the counterion is a halide, such as chloride.

In an embodiment, the transition metal complex can be an osmium transition metal complex that can comprise one or more ligands, wherein at least one (e.g., 1, 2, 3, 4, 5, or 6) ligand can comprise a nitrogen-containing heterocycle (e.g., imidazole, pyrazole, oxazole, thiazole, pyridine, and pyrazine). In some embodiments, the osmium transition metal complex can comprise one or more ligands selected from 4,4′-dimethyl-2,2′-bipyridine, a mono-, di-, or polyalkoxy-2,2′-bipyridine (e.g., 4,4′-dimethoxy-2,2′-bipyridine), 4,7-dimethyl-1,10-phenanthroline, and a mono, di-, or polyalkoxy-1,10-phenanthroline (e.g., 4,7-dimethoxy-1,10-phenanthroline).

In an embodiment, the redox mediator can comprise an osmium complex bonded to a polymer or copolymer of poly(1-vinyl imidazole) or poly(4-vinylpyridine). The poly(4-vinylpyridine)-based polymer can be a prepolymer that has been modified, as shown in the following structure, to attach an osmium complex (e.g., a poly(biimidizyl) osmium complex).

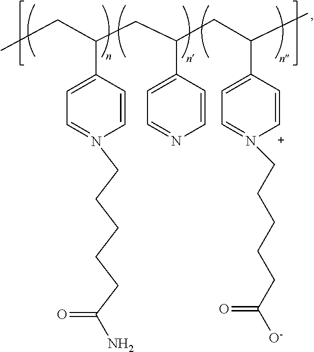

wherein n can be 2, n′ can be 17, and n″ can be 1. Other reactive groups and/or spacer groups can be used.

In an embodiment, the electron redox mediator can comprise an osmium-containing poly(4-vinylpyridine)-based polymer, referred to herein as “X7,” as shown below.

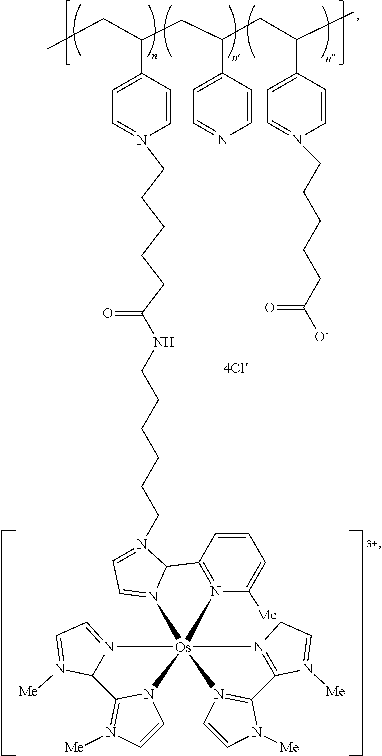

wherein n is 2, n′ is 17, and n″ is 1.

The electron transfer agent typically is attached (e.g., non-leachable and/or covalently bonded) to the polymer in the redox material. For example, covalent bonding of the electron transfer agent to the polymer can take place by polymerizing a monomer unit bearing a covalently bound electron transfer agent, or the electron transfer agent can be reacted with the polymer separately after the polymer has already been synthesized.

According to some embodiments, a bifunctional spacer can be used to attach (e.g., covalently bond) the electron transfer agent to the polymer in the redox material, with a first reactive group being reactive with the polymer (e.g., a functional group capable of quaternizing a pyridine nitrogen atom or an imidazole nitrogen atom) and a second reactive group being reactive with the electron transfer agent (e.g., a functional group that is reactive with a ligand coordinating a metal ion). Typically, covalent bonds are formed between the two reactive groups to generate a linkage. Suitable reactive groups include, for example, activated ester (e.g., succinimidyl, benzotriazolyl, or an aryl substituted with one more electron withdrawing groups, such as sulfo, nitro, cyano, or halo), acrylamido, acyl azido, acyl halide, carboxy (—COO— or —CO₂H), aldehyde, ketone, alkyl halide, alkyl sulfonato, anhydride, aziridino, epoxy, halotriazinyl, imido ester, isocyanato, isothiocyanato, maleimido, sulfonyl halide, amino, thiol (—SH), hydroxy, pyridinyl, imidazolyl, and hydroxyamino. The reaction between two reactive groups can form a covalent linkage between the transition metal complex and the polymer that is a carboxamido, thioether, hydrazonyl, oximyl, alkylamino, ester, carboxylic ester, imidazolium, pyridinium, ether, thioether, aminotriazinyl, triazinyl ether, amidinyl, urea, urethanyl, thiourea, thioether, sulfonamide, or any combination. In addition to the reactive groups, the bifunctional spacer typically can further comprise an alkylenyl (i.e., —(CH₂)ₙ—) and/or ethylenyloxy (i.e., —(CH₂CH₂O)ₘ—, in which n and m are each independently 1 to 12 (e.g., 1 to 11, 1 to 10, 1 to 9, 1 to 8, 1 to 7, 1 to 6, 1 to 5, 1 to 4, 1 to 3, or 1 to 2).

In some embodiments, the sensing composition, and in particular, the redox mediator can further comprise a cross linking agent. In general, the cross linking agent is any suitable multifunctional (e.g., bifunctional) short chain molecule that enables the electron transfer agent to attach (e.g., covalently bond) to the polymer of the redox mediator. For example, the cross linking agent can include a polyepoxide (e.g., a polyethylene glycol diglycidylether (PEGDGE), ethylene glycol diglycidyl ether (EGDGE), resorcinol diglycidyl ether, 1,2,7,8-diepoxyoctane, Gly3), cyanuric chloride, N-hydroxysuccinimide, an imidoester, epichlorohydrin, or a combination thereof. In an embodiment, the cross linking agent is a polyethylene glycol diglycidylether (PEGDGE) of the following formula.

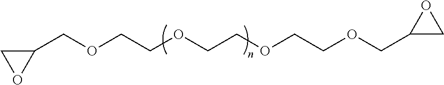

In a particular example, the PEGDGE is PEGDGE200, PEGDGE400 (n is 10), PEGDGE500, PEGDGE600, PEGDGE1000, or PEGDGE2000, in which the number denotes the average molecular weight (Mn). In an embodiment, the crosslinking agent is PEGDGE400.

In an embodiment, at least a portion of the pyruvate-responsive enzyme (e.g., pyruvate oxidase) present in the sensing composition is non-leachably attached to the redox mediator. In some embodiments, the enzyme is covalently attached to the polymer portion of the redox mediator. Covalent bonding of the enzyme to the redox material (e.g., polymer) can take place via the crosslinking agent, as described herein, and a reactive site on the enzyme. Thus, the pyruvate-responsive enzyme (e.g., pyruvate oxidase) is electronically “wired” to a working electrode through the redox material. In an embodiment, a hydrogel is formed upon crosslinking the enzyme and its wire on electrodes. In another embodiment, at least a portion of the pyruvate-responsive enzyme (e.g., pyruvate oxidase) can diffuse into the hydrogel and becomes attached but not necessarily covalently bonded to the polymer.

In some embodiments, the pyruvate-responsive enzyme retains enzymatic (e.g., pyruvate-oxidation) activity when the pyruvate-responsive enzyme is entrained in and/or attached to (e.g., covalently attached to) the polymer. In some embodiments, the pyruvate-responsive enzyme retains at least 70%, at least 80%, at least 90%, at least 95%, at least 97%, at least 98%, at least 99% or more enzymatic (e.g., pyruvate-oxidation) activity of the POX activity when attached to the polymer compared with the enzymatic (e.g., pyruvate-oxidation) activity of the pyruvate-responsive enzyme in solution. Enzymatic (e.g., pyruvate-oxidation) activity can be measured using routine methods known in the art, for example, as utilized in the examples.

In an embodiment, provided is an enzyme sensing composition comprising (i) a pyruvate-responsive enzyme, typically pyruvate oxidase; optionally wherein the composition comprises from about 5 to about 50 mg/mL of the pyruvate-responsive enzyme and (ii) TPP, optionally wherein the composition comprises from about 0.1 to about 20 mg/mL TPP; wherein:
in the composition (a) the weight ratio of the pyruvate-responsive enzyme to TPP is at least 500:1, typically from about 500:1 to about 1:1, more typically from about 200:1 to about 1:1; and/or (b) the molar ratio of the TPP to the pyruvate-responsive enzyme is at least 1:1, typically from about 1.2:1 to about 611.3:1, more typically from about 3.1:1 to about 611.3:1;
the composition typically comprises from about 0.01 to about 1 mg/mL FAD; and/or from about 1 to about 50 mg/mL of an enzyme stabilizer, typically an albumin, more typically BSA or HSA; and/or from about 10 mM to about 500 mM buffer salts and/or from about 10 mM to about 500 mM of one or more Group I or II halide salts and/or has a pH of from about 5 to about 8;
optionally wherein at least a portion of the pyruvate-responsive enzyme present in the sensing composition is attached to or entrained in a redox mediator comprising a polymer and a transition metal complex, typically an osmium complex.

In an embodiment, provided is an enzyme sensing composition comprising
from about 10 to about 30 mg/mL, e.g., about 20 mg/mL, pyruvate oxidase, optionally wherein the pyruvate oxidase is derived from Pseudomonas sp;
from about 0.1 to about 20 mg/mL, e.g. from about 5 mg/mL to about 15 mg/mL TPP;
from about 0.05 to about 0.5 mg/mL FAD;
from about 5 to about 15 mg/mL of an albumin, typically BSA or HSA;
from about 20 mM to about 100 mM buffer salts; typically phosphate; and
from about 20 mM to about 100 mM of one or more Group I or II halide salts, typically NaCl and/or MgCl₂;
   wherein (i) the composition has a pH of from about 6 to about 8, typically about pH 6; and (ii) (a) the weight ratio of the pyruvate oxidase to TPP is from about 10:1 to about 1:1, typically from about 2:1 to about 1:1; and/or (b) the molar ratio of the TPP to the pyruvate oxidase from about 61.1:1 to about 611.3:1, more typically from about 305.7:1 to about 611.3:1;
   optionally wherein at least a portion of the pyruvate-responsive enzyme present in the sensing composition is attached non-leachably to a redox mediator comprising an osmium complex bonded to a polymer or copolymer of poly(1-vinyl imidazole) or poly(4-vinylpyridine).

As described in more detail herein, pyruvate can be sensed using the methods and compositions disclosed herein by electrochemical sensing. Accordingly, in one embodiment is provided an electrode comprising a pyruvate-responsive enzyme (e.g., pyruvate oxidase) as described herein, and TPP. The ratio of the pyruvate-responsive enzyme (e.g., pyruvate oxidase) to TPP can be as described herein. The electrode can also comprise other components of the sensing composition described herein. In one embodiment, therefore, is provided an electrode comprising an enzyme sensing composition, as described herein, typically wherein the composition is typically comprised in a sensing layer on a working electrode.

Also provided herein is a method of producing an electrode for sensing pyruvate, comprising disposing on the surface of the electrode a sensing composition as described in more detail herein. In some embodiments the method further comprises incorporating the electrode into a sensor or other apparatus as described herein.

The present disclosure further relates to a sensing electrode, which can comprise a working electrode comprising a pyruvate sensing layer formed from (or comprising) the sensing composition as described herein. The working electrode can be any suitable conductive material, such as carbon, gold, palladium, or platinum. The pyruvate sensing layer can be continuous or discontinuous (e.g., forming a spot, a line, or a plurality (i.e., an array) of spots and/or lines). The number of spots is not considered to be particularly limited, but can range from 2 to about 10 (e.g., about 3 to about 8, or from about 4 to about 6). In an embodiment, the pyruvate sensing layer can be continuous. In other embodiments, the pyruvate sensing layer can be discontinuous.

The total size of the sensing layer or layers (combined area of all spots or layers) can be at least about 0.05 mm² and can be up to about 100 mm². In some embodiments, the total size can be about 100 mm² or less, about 75 mm² or less, about 50 mm² or less, about 40 mm² or less, about 30 mm² or less, about 25 mm² or less, about 15 mm² or less, about 10 mm² or less, about 5 mm² or less, about 1 mm² or less, or about 0.1 mm² or less. In a particular embodiment, the total size of the sensing layer or layers ranges from about 0.05 to about 0.1 mm², about 0.05 to about 100 mm², about 0.1 to about 50 mm², about 0.5 to about 30 mm², about 1 to about 20 mm², or about 1 to about 15 mm².

The sensing layer or layers typically have a thickness that ranges from about 0.1-10 μm. For example, each sensing layer should be 0.1 μm thick or more (e.g., 0.2 μm or more, 0.3 μm or more, 0.5 μm or more, 0.8 μm or more, 1 μm or more, 2 μm or more, 3 μm or more, 5 μm or more, or 8 μm or more) and typically will have a thickness of 10 μm or less (e.g., 8 μm or less, 5 μm or less, 3 μm or less, 2 μm or less, 1 μm or less, 0.8 μm or less, 0.5 μm or less, 0.3 μm or less, or 0.2 μm or less). In an example, each sensing layer present has a thickness of about 0.1 to about 10 μm, about 0.2 to about 8 μm, about 0.5 to about 5 μm, about 1 to about 4 μm, or about 2 μm.

In some embodiments, a conductive material such as, for example, carbon nanotubes, graphene, or metal nanoparticles, can be combined within the sensing layer or layers to promote rapid attainment of a steady state current. Conductive material can be included in a range from about 0.1% to about 50% by weight (pbw) of the sensing layer (e.g., about 1 to about 50 pbw, about 1 to about 10 pbw, or about 0.1 to about 10 pbw).

In an embodiment, the sensing electrode can further comprise a membrane that overcoats at least the pyruvate sensing layer and optionally other components. The overcoating forms an outer membrane that provides stability to the sensing reagents (e.g., the analyte-specific enzyme and redox mediator), mass-transport limitations, biocompatibility, and/or prevents electrode fouling. The membrane can optionally coat all or part of the working electrode and optionally any counter or reference electrode that can be present. In an embodiment, the membrane coats (e.g., encapsulates) the entire system, including the sensing electrode with its sensing layer(s), and any counter electrodes, reference electrodes, and/or substrates that can be present.

The membrane can comprise one or more polymeric membrane materials with a physical structure that allows analyte flux to the sensing layer (i.e., the membrane is a mass transport limiting membrane). The composition of the membrane can vary (e.g., the degree of hydrophobicity and/or the degree of crosslinking) to promote a desired flux of pyruvate (and any other additional analytes) to the sensing electrode, thereby providing a desired signal intensity and stability as described further herein. In an embodiment, the membrane is permeable to pyruvate. In general, serum contains a low concentration of pyruvate (e.g., about 80 to about 160 μM), such that a permeable membrane enables the detection of pyruvate. In an embodiment, the membrane has sufficient pyruvate permeability to provide an analyte sensitivity of about 1 nA/mM or greater when exposed to pyruvate. In this or other embodiments, the membrane has reduced permeability to TPP relative to pyruvate. Such reduced permeability minimizes the amount of TPP that leaches from the pyruvate sensing layer through the membrane. In an embodiment, the membrane is hydrophobic.

The coating of the membrane over at least the pyruvate sensing layer can be performed by any suitable technique. Typically, the membrane will be coated by spray coating, painting, inkjet printing, roller coating, dip coating, or any combination thereof. The coating step can be performed once or multiple times (e.g., 2, 3, 4, or 5 times), which will affect the thickness of the membrane coating. In an embodiment, the coating step can be performed twice to form a bilayer.

In general, if multiple coats are applied, the first coat will be dried prior to applying the subsequent coat(s). The amount of time between coating steps will vary depending on the types of membrane, working electrode, and sensing layer, and the atmospheric conditions. In general, the drying time will be 1 minute or longer (e.g., 2 min or more, 3 min or more, 5 min or more, 10 min or more, 15 min or more, or 20 min or more). Once the membrane coating has been applied, the coating can be cured. In an embodiment, the coating can be cured for 12 hours or more (e.g., 18 hours or more, 24 hours or more, 30 hours or more, 36 hours or more, 42 hours or more, or 48 hours or more). The curing can be at room temperature (i.e., about 20° C.) or at a slightly elevated temperature (e.g., 100° C. or less, 80° C. or less, 70° C. or less, 60° C. or less, 50° C. or less, 40° C. or less, 30° C. or less, or 25° C. or less). In general, the curing will not occur at less than about 20° C.

The membrane typically has a thickness that ranges from about 1 μm to about 100 μm. For example, in some embodiments, the membrane can have a thickness of about 1 μm or more (e.g., about 5 μm or more, about 10 μm or more, about 15 μm or more, about 20 μm or more, about 25 μm or more, about 30 μm or more, about 35 μm or more, about 40 μm or more, about 50 μm or more, about 60 μm or more, about 70 μm or more, about 80 μm or more, or about 90 μm or more) and typically will have a thickness of about 100 μm or less (e.g., about 90 μm or less, about 80 μm or less, about 70 μm or less, about 60 μm or less, about 50 μm or less, about 45 μm or less, about 40 μm or less, about 35 μm or less, about 30 μm or less, about 25 μm or less, about 20 μm or less, about 15 μm or less, about 10 μm or less, or about 5 μm or less). In an example, the membrane can have a thickness of about 5 to about 80 μm, about 10 to about 80 μm, about 10 to about 60 μm, about 15 to about 60 μm, about 20 to about 50 μm, about 20 to about 40 μm, about 25 to about 35 μm, or a thickness of about 30 μm. In an example, the membrane can have a thickness of about 15 to about 35 μm. In an example, the membrane can have a thickness of about 20 to about 35 μm. In an example, the membrane can have a thickness of about 20 to about 30 μm. In an example, the membrane can have a thickness of about 25 μm.

In an embodiment, the membrane can comprise optionally crosslinked poly(4-vinylpyridine), poly(vinyl alcohol), poly(acrylic acid), poly(methacrylic acid), or a combination thereof. In an example, the mass transport limiting membrane can comprise at least a poly(4-vinylpyridine) homopolymer or copolymer, in which the poly(4-vinylpyridine) can be optionally cross linked. Particularly, the membrane can comprise poly(4-vinylpyridine) crosslinked with a crosslinking agent, as described herein (e.g., high molecular weight (such as molecular weight 400 g/mol) poly(ethylene glycol) diglycidyl ether).

Suitable poly(4-vinylpyridine) copolymers for inclusion in the mass transport limiting membrane can comprise up to about 25% comonomers (based on the total amount of monomers in the copolymer), such as from about 0.1% to about 5% comonomers, or about 5% to about 15% comonomers, or about 15% to about 25% comonomers, or about 1% to about 10% co-monomers. Suitable comonomers are not particularly limited, provided that the mass transport limiting membrane affords sufficient pyruvate permeability to provide an analyte sensitivity of about 1 nA/mM or greater when exposed to pyruvate.

In some embodiments, the membrane can comprise multiple layers in which each layer has a different composition and/or degree of crosslinking. In an example, the membrane coating can be a bilayer membrane that can comprise a first layer formed from a poly(4-vinylpyridine) homopolymer or copolymer and a second layer formed from a crosslinked poly(4-vinylpyridine) homopolymer or copolymer (e.g., crosslinked with PEGDGE). The variation in composition between layers allows tuning the permeability of the membrane to both pyruvate and TPP.

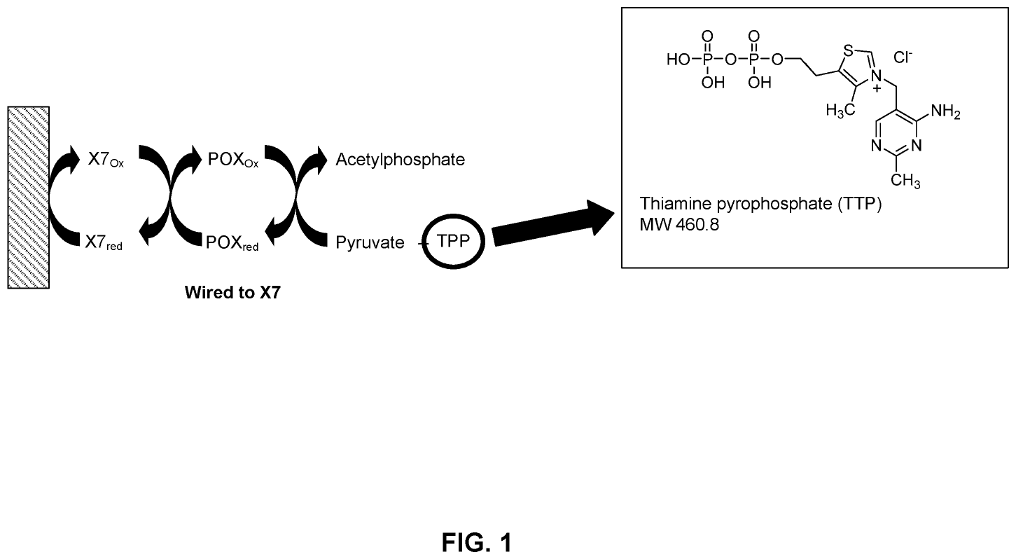

The present disclosure also relates to a system for sensing pyruvate. The system can comprise a working electrode, a sensing element disposed on at least a portion of the working electrode, and a circuit configured to connect and disconnect with the working electrode. The sensing element can comprise the sensing composition as described herein, and the sensing element is configured to accumulate charge derived from pyruvate reacting with the pyruvate-responsive enzyme (e.g., pyruvate oxidase) for a set period of time. In an embodiment, the system can be a sensor (e.g., an enzymatic biosensor). In an example, the sensor chemistry is described in FIG. 1, in which X7 is the redox material.

In some embodiments, the disclosed systems and methods are for sensing pyruvate in a biological sample. Samples include both biological and environmental samples. Samples can be detected in a laboratory setting, in the field, or any other suitable location. The samples can be brought to a sensor as described herein for testing, or the sensor can be applied at the source of the samples.

Biological samples can be obtained from any source including animals, plants, and microorganisms and encompass fluids, solids, tissues, and gases. Materials obtained from clinical or forensic settings that contain analytes of interest are also within the intended meaning of the term sample. Biological samples include, but are not limited to, whole blood, serum, plasma, saliva, ocular lens fluid, amniotic fluid, synovial fluid, cerebrospinal fluid, lacrimal fluid, lymph fluid, interstitial fluid, peritoneal fluid, bronchial lavage, ascites fluid, bone marrow aspirate, pleural effusion, urine, milk, sweat, sputum, semen, mucus, feces, tissue (skeletal muscle tissue, liver tissue, lung tissue, kidney tissue, myocardial tissue, brain tissue, bone marrow, cervix tissue, skin, etc.), organ (such as biopsy sample), vaginal fluids, aqueous humor, earwax, gastric fluid, gastrointestinal fluid, nasal wash, liposuction, sebum, tears, breath, and vitreous humor. Such samples may be assessed in vitro, ex vivo, or in vivo.

In some embodiments, samples can be in a processed form, including dried (e.g., dried blood spots) and fixed (e.g., formalin-fixed paraffin-embedded (FFPE)) samples. In some embodiments, the sample is located in vivo in an animal.

Since pyruvate is present in a relatively low concentration in biofluid (e.g., serum), the system is designed to detect a low concentration of analyte by allowing for an accumulation of the analyte on an enzymatic biosensor. In the context of detecting pyruvate, a low concentration can be about 1 mM or less (e.g., 900 μM or less, 800 μM or less, 700 μM or less, 600 μM or less, 500 μM or less, 400 μM or less, 300 μM or less, 200 μM or less, or 100 μM or less) and 10 μM or more (e.g., 20 μM or more, 30 μM or more, 40 μM or more, 50 μM or more, 60 μM or more, 70 μM or more, 80 μM or more, 90 μM or more, 100 μM or more, 120 μM or more, 140 μM or more, or 150 μM or more). For example, the pyruvate concentration in a sample or analyte can be about 10 μM to about 1 mM, about 50 to about 400 μM, about 60 to about 300 μM, or about 70 to about 200 μM. In some embodiments, the methods disclosed herein are methods for detecting pyruvate concentrations of from about 10 μM to about 1 mM, about 50 to about 400 μM, about 60 to about 300 μM, or about 70 to about 200 μM, such as from about 80 to about 160 μM.

In some embodiments, therefore, provided here is a system comprising a pyruvate-responsive enzyme, as disclosed herein, such as pyruvate oxidase; TPP; and a sample comprising pyruvate, wherein the ratio (e.g., weight ratio) of the pyruvate-responsive enzyme to TPP is as described herein (e.g., is a weight ratio of from about 500:1 to about 1:1). In some embodiments, the pyruvate is present in a biological sample as described herein. In some embodiments, the pyruvate is present in the sample in a concentration of from about 1 μM to about 1 mM.

In an embodiment, the sensor can comprise a working electrode and another electrode (e.g., a counter electrode and/or reference electrode), in which the working electrode is provided with a sensing element disposed on at least a portion of the working electrode. Other components, such as a substrate can be present, if needed. The substrate (e.g., a carbon-based substrate, a plastic substrate) can be disposed between the working electrode and a counter and/or reference electrode. The sensing element can be formed from a sensing composition, as described herein, e.g., can comprise at least a pyruvate-responsive enzyme (e.g., pyruvate oxidase), TPP, FAD, an albumin, and a redox mediator. The working electrode that includes the sensing element forms a sensing electrode. In the presence of analyte, the sensing electrode oxidizes the analyte, and the amount of oxidation is measured as the amount of electron charge produced from the reaction. As long as the sensing electrode is not connected to another electrode, the charge from the redox reaction will continue to accumulate on the sensing electrode. Accumulating charge (electrons) for a set period of time allows for low concentrations of pyruvate to result in a signal output that is easy to measure and quantify compared to other known methods. After a set period of time for charge accumulation, the sensing electrode is connected with at least one (e.g., 1, 2, 3, or 4) other electrode such as a counter electrode and/or reference electrode to form a circuit. Upon connecting the circuit, the accumulated electrons on the sensing electrode are discharged as an electrical signal, the amplitude of which is measured and correlates to the amount of pyruvate present at the sensing electrode.

The sensing of the analyte (A) relies on having an oxidoreductase enzyme (AOx) electrically “wired” to the working electrode of the sensor through a redox material. During normal amperometric sensing, the electrode is poised at a potential (voltage) so that the analyte is reacted at a constant rate, which is proportional to the analyte concentration. For an analyte oxidation reaction (A to A+), the electrons will flow from the analyte (A) to the analyte-specific enzyme (AOx) to the redox material (e.g., Os³⁺) to the working electrode at a constant rate, producing a steady-state current. If the working electrode is disconnected from the circuit, the flow of electrons from the redox polymer to the working electrode will stop, resulting in no current flow through the circuit. However, the analyte will still undergo enzymatic oxidation, which in turn results in reducing the redox material (e.g., Os³⁺ to Os²⁺). This results in a buildup of the reduced form of the redox material (e.g., Os²⁺) over time, as electrons (e) from the analyte are stored in the redox material. When the working electrode is reconnected to the circuit so that it is poised at its original potential (voltage), the buildup of the reduced form of the redox material will be oxidized, resulting in a large current spike. The current will then decay back to the original amperometric current as the redox system reaches steady-state once again. This two-step process forms the basis for accumulation mode sensing: one in which the working electrode of the sensor is disconnected from or not connected to the circuit for a set period of time (also referred to as the accumulation time), enabling charge from the analyte to “accumulate” in the redox polymer, and a second in which the working electrode of the sensor is connected to the circuit after the accumulation time, enabling the accumulated charge to be discharged and measured as a sharp peak.

In an example of a three electrode set-up, there can be a sensing electrode (i.e., a working electrode with a pyruvate sensing layer), a reference electrode, and a counter electrode used for accumulation mode sensing in which when the circuit is connected, the sensing electrode is poised at a potential (voltage) sufficient to drive the redox reaction of the analyte under steady-state conditions. For example, for the pyruvate sensor described herein, the potential (voltage) sufficient to drive the redox reaction is +40 mV vs. Ag/AgCl. When the circuit is not connected, the working electrode is electrically disconnected from the circuit, enabling charge (e.g., electrons) from the analyte to be stored in the redox material until the sensing electrode is reconnected to the circuit and the stored charge is measured. In an aspect, the counter electrode can be carbon (e.g., screen-printed carbon), and the reference electrode can be Ag/AgCl. In a two electrode example, a sensing electrode and a second electrode that functions as both a counter electrode and reference electrode (i.e., a counter/reference electrode) are used.

In some embodiments, the set period of time can be any suitable time period that allows for accumulating adequate charge to detect pyruvate once the circuit is reconnected. Typically, the set period of time can be about 30 seconds or more (e.g., 1 minute or more, 2 minutes or more, 3 minutes or more, 4 minutes or more, 5 minutes or more, 6 minutes or more, 7 minutes or more, 8 minutes or more, 10 minutes or more, 15 minutes or more, 20 minutes or more, 25 minutes or more). In general, the set period of time can be about 30 minutes or less (e.g., 25 minutes or less, 20 minutes or less, 15 minutes or less, 10 minutes or less, 8 minutes or less, 7 minutes or less, 6 minutes or less, 5 minutes or less, 4 minutes or less, 3 minutes or less, 2 minutes or less, or 1 minute or less) to allow for complete reaction of all of the pyruvate present at the sensing electrode. For example, the set period of time can be about 30 seconds or more and about 30 minutes or less, about 1 to about 20 minutes, about 1 to about 15 minutes, about 1 to about 10 minutes, about 2 to about 8 minutes, about 2 to about 5 minutes, or about 30 seconds or more. In some embodiments the set period of time can be between about 30 s and about 1 hour, such as between about 1 minute and about 45 minutes, such as between about 2 minutes and about 30 minutes, e.g. about 5 minutes to about 15 minutes.

Accordingly, in some embodiments, provided is a method for sensing pyruvate comprising: contacting a sample comprising pyruvate with a sensing electrode as described herein; and detecting accumulated charge derived from pyruvate reacting with the pyruvate oxidase and the redox mediator.

In some embodiments, a sensor disclosed herein is configured to penetrate the skin of a subject. The sensor can comprise a member capable of penetrating the skin of a subject. For example, the member can be an insertable tip, tail, probe or needle capable of penetrating the skin of a subject. In some embodiments, the methods disclosed herein are for detecting subcutaneous and/or interstitial pyruvate concentrations.

In an embodiment, the system (e.g., sensor) can further comprise a sensor tail (e.g., insertion tip, implantable portion) configured for penetrating (e.g., implantation into) a tissue, wherein the working electrode is disposed on the sensor tail (e.g., insertion tip, implantable portion). In general, the sensor tail (e.g., insertion tip, implantable portion) can be of sufficient size and shape to be positionable below the surface of the tissue (e.g., penetrating through the skin (dermis)) and into the subcutaneous space and in contact with the wearer's biofluid, such as interstitial fluid. In an example, a sensor tail (e.g., insertion tip, implantable portion) can be about 5 mm in length, about 0.6 mm in width, and about 0.25 mm in thickness. Suitable tissues include, for example, skin, including the dermal layer, an interstitial layer, and/or a subcutaneous layer of the skin.

In an example, electrode contacts are positioned on a first portion of the sensor situated above the skin surface and extend to a location in the sensor tail (e.g., insertion tip, implantable portion). A working electrode, a reference electrode, and a counter electrode are at a second portion of the sensor, typically at a bottom portion of the sensor tail (e.g., insertion tip, implantable portion). The working electrode will comprise a pyruvate sensing layer and can optionally comprise a sensing layer for an analyte other than pyruvate, as described herein.

The system (e.g., sensor) can further be contained within a sensor housing that is configured for adherence to the tissue (e.g., skin). If necessary, the sensor housing can include an adhesive layer that enables adhesion to the desired tissue. The sensor housing can hold all necessary components of the sensor, such as circuitry and a power source for operating the sensor. In some embodiments, the power source (e.g., a coin cell battery) and/or active circuitry are not contained within the sensor housing. A processor can be communicatively coupled to the sensor, in which the processor is physically located within the sensor housing or a reader device. The power source can include one or more batteries, which can be rechargeable or single-use disposable batteries. Power management circuitry can regulate battery charging and power supply monitoring, boost power, or perform direct current (DC) conversions.

The present disclosure is also directed to a method for sensing pyruvate. In an embodiment, the method can comprise contacting a sample comprising pyruvate with the sensing electrode as described herein; and detecting an accumulated charge derived from pyruvate reacting with the pyruvate oxidase and the redox mediator. In some embodiments, the method can comprise: providing the sensing electrode as described herein, connecting the sensing electrode to a circuit to provide a steady state, disconnecting the sensing electrode from the circuit, contacting the sensing electrode to an analyte that can comprise pyruvate, accumulating charge derived from pyruvate reacting with the pyruvate-responsive enzyme (e.g., pyruvate oxidase) and the redox mediator for a set period of time, connecting the sensing electrode to a circuit after the set period of time; and measuring a signal from the accumulated charge.

As described herein, the set period of time for the method can be any suitable time period that allows for accumulating adequate charge to detect pyruvate once the circuit is reconnected. In an embodiment, the set period of time can be 30 seconds or more.

In general, the method uses a system (e.g., a sensor), as disclosed herein, for measuring low concentrations of pyruvate and can be used in an in vivo monitoring system, which while positioned in vivo in a user (e.g., a patient, such as a human) makes contact with the biofluid of the user and senses one or more analyte levels contained therein. An in vivo monitoring system can include one or more reader devices that receives sensed analyte data from a sensor control device. The reader device can process and/or display the sensed analyte data or sensor data in any number of forms to the user. In some embodiments, the reader device can be a mobile communication device, such as a dedicated reader device (configured for communication with a sensor control device) optionally in conjunction with a computer system, a mobile telephone (e.g., a WiFi or internet-enabled smart phone), a tablet, a personal digital assistant (PDA), or a mobile smart wearable electronics assembly (e.g., a smart glass, smart glasses, watch, bracelet, or necklace). Configuring a reader device to an in vivo monitoring system is described at, for example, U.S. Pat. No. 11,371,957, the disclosure of which is incorporated herein by reference in its entirety.

The reader device typically includes an input component, a display, and processing circuitry, which can include one or more processors, microprocessors, controllers, and/or microcontrollers, each of which can be a discrete chip or distributed amongst (and a portion of) a number of different chips. The processing circuitry can include a communications processor having on-board memory and an applications processor having on-board memory. The reader device can further include radio frequency (RF) communication circuitry coupled with an RF antenna, a memory, multi-functional circuitry with one or more associated antennas, a power supply, power management circuitry, and/or a clock. It will be recognized that other hardware and functionality can be included in the reader device.

Sleep monitoring is increasingly attracting attention due to the associations between sleep and both physical and mental health and wellbeing. Pyruvate has been associated with sleep quality, with pyruvate levels being increased during REM sleep cycles and reduced during periods of wakefulness. Monitoring pyruvate levels in a subject, or in a sample obtained from a subject, could thus allow sleep (e.g., both sleep quality and sleep quantity) to be tracked or monitored. Information from sleep monitoring is useful for many individuals, such as those seeking to improve their sleep quality (e.g., individuals with sleep disorders such as insomnia) and/or individuals with mental or physical disorders which are characterized by poor sleep, where monitoring sleep quality can inform the individual about the underlying condition and the efficacy or otherwise of treatments for such.

Weight loss is another area in which pyruvate monitoring is valuable. Pyruvate has been associated with lipogenesis and fat metabolism in vivo. Accordingly, monitoring pyruvate levels in a subject, or in a sample obtained from a subject, could provide information regarding the health status of the subject. Monitoring pyruvate levels could inform dietary and lifestyle choices made by an individual.

Pyruvate and lactate levels also fluctuate during exercise. Monitoring pyruvate in a subject, or in a sample obtained from a subject, could thus provide information regarding the health and wellness of the subject, including recovery rates following exercise.

As described above, pyruvate sensing is useful in determining and monitoring various health and wellness conditions, including chronic, progressive diseases such as chronic obstructive pulmonary disease (COPD), obesity, diabetes, and aging; sleep quality and quantity; weight loss and diet; and fitness and recovery from exercise. Information about such can be provided by detecting and/or monitoring the presence, absence or concentration of pyruvate in a sample from a subject.

Accordingly, in some embodiments, provided is method of detecting the presence, absence or concentration of pyruvate in a sample, comprising: contacting the sample with an electrode as described herein; and taking one or more measurements characteristic of pyruvate oxidation by the pyruvate-responsive enzyme in the sensing composition of the electrode.

In some embodiments the electrode is present in a system and/or device as described herein.

Further provided is the use of a pyruvate-responsive enzyme, optionally pyruvate oxidase, and TPP, to detect the presence, absence or concentration of pyruvate in a sample, wherein the weight ratio of the pyruvate-responsive enzyme to TPP is from about 500:1 to about 1:1. The use may be further as described herein.

Also provided is a method of determining the health and/or wellbeing of a subject, comprising contacting a biological sample from said subject with a sensor as described herein; and determining the health and/or wellbeing of the subject. In some embodiments determining the health and/or wellbeing of the subject comprises monitoring and/or detecting one or more of chronic, progressive diseases such as chronic obstructive pulmonary disease (COPD), obesity, diabetes, and aging; sleep quality and quantity; weight loss and diet; and fitness and recovery from exercise.

The present disclosure is further illustrated by the following embodiments.
(1) A sensing composition comprising a pyruvate-responsive enzyme, thiamine pyrophosphate (TPP), flavin adenine dinucleotide (FAD), an albumin, and a redox mediator.
(2) The sensing composition of embodiment 1, wherein the pyruvate-responsive enzyme comprises pyruvate oxidase.
(3) The sensing composition of embodiment 1 or 2, wherein the pyruvate oxidase and TPP are present in the composition in a weight ratio of about 500:1 to about 1:1.
(4) The sensing composition of any one of embodiments 1-3, wherein the albumin is bovine serum albumin.
(5) The sensing composition of any one of embodiments 1-4, further comprising a pH buffer.
(6) The sensing composition of any one of embodiments 1-5, wherein the redox mediator comprises a polymer and an electron transfer agent.
(7) The sensing composition of embodiment 6, wherein the polymer comprises poly(4-vinylpyridine), poly(1-vinylimidazole), poly(thiophene), poly(aniline), poly(pyrrole), or poly(acetylene).
(8) The sensing composition of embodiment 6, wherein the polymer comprises a polymer or copolymer repeat unit comprising at least one pendant pyridinyl group, imidazolyl group, or both a pyridinyl and imidazolyl group.
(9) The sensing composition of any one of embodiments 6-8, wherein the electron transfer agent comprises a transition metal complex.
(10) The sensing composition of embodiment 9, wherein the transition metal complex comprises osmium, ruthenium, iron, cobalt, vanadium, or a combination thereof.
(11) The sensing composition of embodiment 9 or 10, wherein the transition metal complex is an osmium transition metal complex comprising one or more ligands, wherein at least one ligand comprises a nitrogen-containing heterocycle.
(12) The sensing composition of any one of embodiments 1-11, wherein the redox mediator comprises an osmium complex bonded to a poly(4-vinylpyridine)-based polymer.
(13) The sensing composition of any one of embodiments 1-12, further comprising a cross linking agent.
(14) The sensing composition of embodiment 13, wherein the cross linking agent is a polyepoxide, cyanuric chloride, N-hydroxysuccinimide, an imidoester, epichlorohydrin, or a combination thereof.
(15) The sensing composition of embodiment 13 or 14, wherein the cross linking agent is a polyethylene glycol diglycidylether (PEGDGE).
(16) The sensing composition of any one of embodiments 1-15, wherein the pyruvate oxidase is attached to the redox mediator.
(17) A sensing electrode comprising a working electrode comprising a pyruvate sensing layer formed from the sensing composition of any one of embodiments 1-16.
(18) The sensing electrode of embodiment 17, wherein the pyruvate sensing layer is continuous.
(19) The sensing electrode of embodiment 17, wherein the pyruvate sensing layer is discontinuous.
(20) The sensing electrode of any one of embodiments 17-19, further comprising a membrane overcoating at least the pyruvate sensing layer.
(21) The sensing electrode of embodiment 20, wherein the membrane is permeable to pyruvate.
(22) The sensing electrode of embodiment 20 or 21, wherein the membrane has reduced permeability to TPP relative to pyruvate.
(23) The sensing electrode of any one of embodiments 20-22, wherein the membrane comprises poly(4-vinylpyridine).
(24) A system for sensing pyruvate comprising a working electrode, a sensing element disposed on at least a portion of the working electrode, and a circuit configured to connect and disconnect with the working electrode, wherein the sensing element comprises the sensing composition of any one of embodiments 1-15, and the sensing element is configured to accumulate charge derived from pyruvate reacting with the pyruvate oxidase for a set period of time.
(25) The system of embodiment 24, further comprising a sensor tail configured for implantation into a tissue, wherein the working electrode is disposed on the sensor tail.
(26) A method for sensing pyruvate comprising: providing the sensing electrode of any one of embodiments 17-23; connecting the sensing electrode to a circuit to provide a steady state; disconnecting the sensing electrode from the circuit; contacting the sensing electrode to an analyte comprising pyruvate; accumulating charge derived from pyruvate reacting with the pyruvate oxidase and the redox mediator for a set period of time; connecting the sensing electrode to a circuit after the set period of time; and measuring a signal from the accumulated charge.
(27) The method of embodiment 26, wherein the set period of time is 30 seconds or more.
(28) A method of producing an electrode for sensing pyruvate, comprising disposing on the surface of an electrode a sensing composition described herein (e.g., any one of embodiments 1-16).
(29) A system comprising:
a pyruvate-responsive enzyme, optionally pyruvate oxidase;
TPP; and
a sample comprising pyruvate; optionally wherein the pyruvate is present in the sample in a concentration of from about 1 μM to about 1 mM; wherein the weight ratio of the pyruvate-responsive enzyme to TPP is from about 500:1 to about 1:1.
(30) A method of detecting the presence, absence or concentration of pyruvate in a sample, comprising:
contacting the sample with a sensing electrode described herein (e.g., any one of embodiments 17-23), optionally wherein the sensing electrode is comprised in a system according to embodiment 29; and
taking one or more measurements characteristic of pyruvate oxidation by the pyruvate-responsive enzyme in the sensing composition of the electrode.
(31) A method of determining the health and/or wellbeing of a subject, comprising
contacting a biological sample from said subject with a sensor described herein (e.g., any one of embodiments 1-16); and
determining the health and/or wellbeing of the subject.
(32) Use of a pyruvate-responsive enzyme, optionally pyruvate oxidase, and TPP, to detect the presence, absence, or concentration of pyruvate in a sample, wherein the weight ratio of the pyruvate-responsive enzyme to TPP is from about 500:1 to about 1:1.

### EXAMPLES

These examples are provided for the purpose of illustration only and the embodiments described herein should in no way be construed as being limited to these examples. Rather, the embodiments should be construed to encompass any and all variations which become evident as a result of the teaching provided herein.

### Example 1

A pyruvate oxidase (POX) containing sensing layer was deposited using a solution formulated as specified in Table 1 below. FAD and TPP were present as cofactors, along with a phosphate buffer, bovine serum album (BSA), the osmium-based redox material known as X7, and crosslinking agent PEGDGE400. Two different concentrations of TPP were tested. One sample contained a pyruvate oxidase (from Pseudomonas sp., Toyobo USA) to TPP weight ratio of 200:1 (“1×TPP”; corresponding to a molar ration of TPP:POX of 3.1:1), and a second sample that contained over 100 times more TPP than the first sample to provide a pyruvate oxidase to TPP weight ratio of 1.73:1 (“100×TPP”; corresponding to a molar ratio of TPP:POX of 353.4:1). Each solution of the sensing composition was applied to a carbon electrode to provide a continuous pyruvate sensing layer. Each sensing layer was subsequently cured overnight at 25° C.

A mass transport limiting membrane was coated over the electrode with the pyruvate sensing layer using an alcohol-buffer solution of the materials specified in Table 2. Dip coating was used to deposit the membrane upon the pyruvate-sensing layer prepared as above. The membrane solution was deposited using 2 dips, and a wait time of about 10 minutes between dips was used. Following the completion of dip coating, the membrane was cured for 24 hours at 25° C., followed by 48 hours at 56° C. in desiccated vials to provide sensing electrodes. Spray coating, screen printing, or similar processes can also be used to deposit the mass transport limiting membrane over at least the sensing layer. The membrane thickness was determined to be about 18 μm.

TABLE 1

| | |
|---|---|
| | Final |
| Reagents | mg/mL |

| | | |
|---|---|---|
| Buffer* | 1 × TPP | 100 × TPP |
| POX (from Toyobo) | 20.0 | 20.0 |
| FAD (from Sigma) | 0.1 | 0.1 |
| TPP (from Sigma) | 0.1 | 11.5 |
| BSA (from Sigma) | 10.0 | 10.0 |
| X7 | 10.0 | 10.0 |
| PEGDGE400 | 5.0 | 5.0 |

*50 mM phosphate, 50 mM NaCl, 10 mM MgCl₂, pH 6.0

TABLE 2

| Reagents | Final |
|---|---|
| EtOH/HEPES buffer (80/20) | mg/mL |
| poly(4-vinylpyridine) (PVP) | 96 |
| PEGDGE400 | 4 |

### Example 2

The sensing electrodes prepared in Example 1 were tested in a system to measure the detection signal (nA) at various pyruvate concentrations over time. The system was tested using an accumulating charge method. In particular, the sensing electrode was connected to a circuit to provide a steady state. The sensing electrode was then disconnected from the circuit. The sensing electrode was contacted with various samples comprising different concentrations of pyruvate (0 μM, 50 μM, 100 μM, 150 μM, and 200 μM). Charge derived from pyruvate reacting with the pyruvate oxidase and the redox mediator (X7) was accumulated for a set 30 minute period of time while the sensing electrode was still disconnected from the circuit. The sensing electrode was then reconnected to the circuit after the set period of time; and the signal from the accumulated charge was measured.

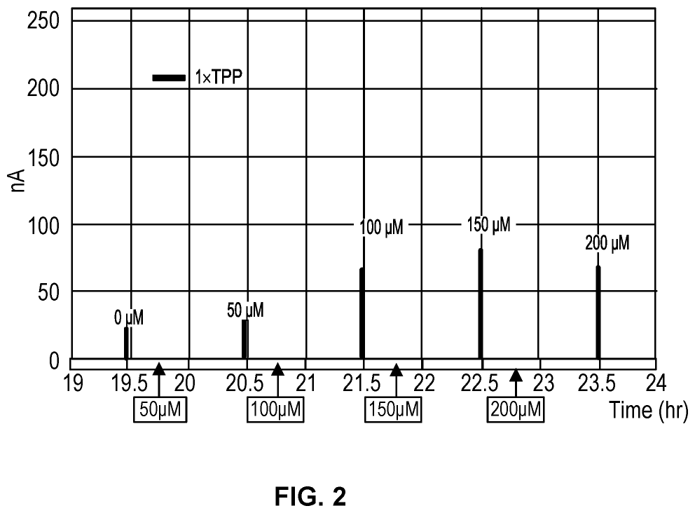

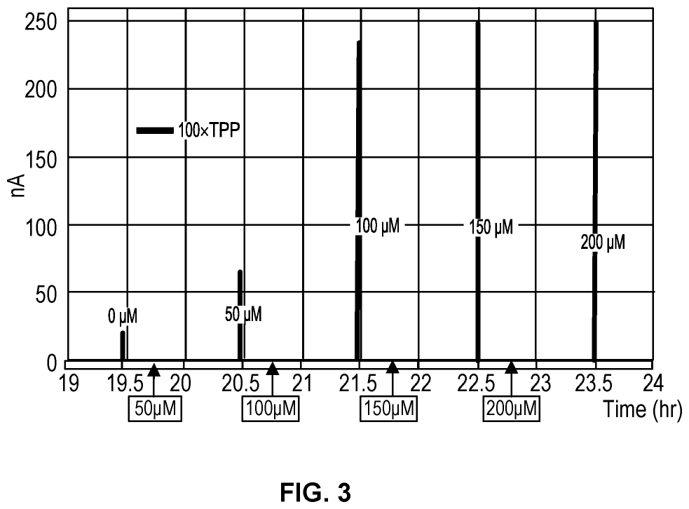

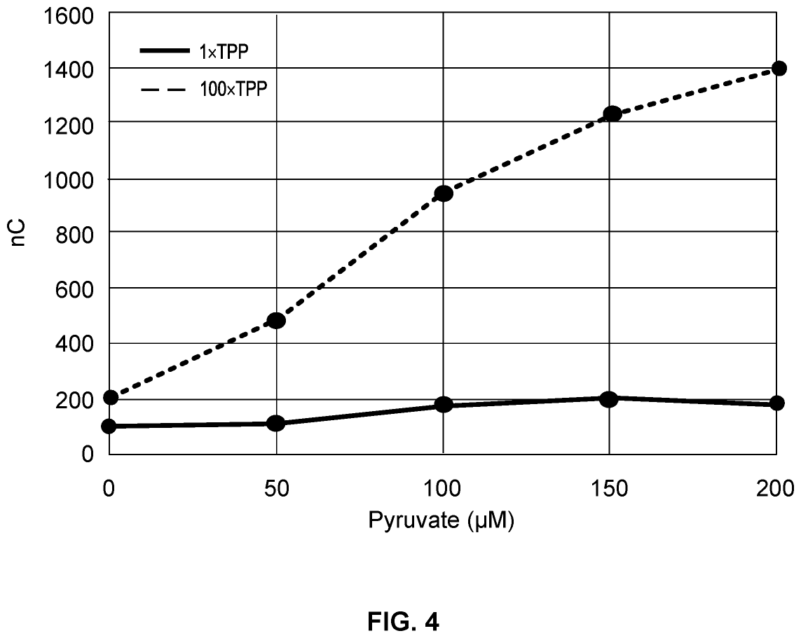

The results for the 1×TPP composition and 100×TPP composition are shown in FIGS. 2 and 3, respectively. With these data, accumulated charge (as measured in nanocoulombs) was plotted versus the pyruvate concentration (μM) are shown in FIG. 4. FIG. 4 demonstrates that the pyruvate signal is improved using an accumulation detection technique when the TPP quantity is increased relative to pyruvate oxidase.

It is to be appreciated that the Detailed Description section, and not the Summary and Abstract sections, is intended to be used to interpret the claims. The Summary and Abstract sections may set forth one or more but not all exemplary embodiments of the present invention as contemplated by the inventor(s), and thus, are not intended to limit the present invention and the appended claims in any way.

The present disclosure has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present invention should not be limited by any of the above-described exemplary embodiments, but should be defined only in accordance with the following claims and their equivalents.

The claims in the instant application are different than those of the parent application or other related applications. The Applicant therefore rescinds any disclaimer of claim scope made in the parent application or any predecessor application in relation to the instant application. The Examiner is therefore advised that any such previous disclaimer and the cited references that it was made to avoid, may need to be revisited. Further, the Examiner is also reminded that any disclaimer made in the instant application should not be read into or against the parent application.

1. A sensing composition comprising a pyruvate-responsive enzyme, thiamine pyrophosphate (TPP), optionally flavin adenine dinucleotide (FAD), a stabilizing agent, and a redox mediator.

2. The sensing composition of claim 1, wherein the pyruvate-responsive enzyme comprises pyruvate oxidase.

3. The sensing composition of claim 2, wherein the pyruvate oxidase and TPP are present in the composition in a weight ratio of about 500:1 to about 1:1.

4. The sensing composition of claim 3, wherein the pyruvate oxidase and TPP are present in the composition in a weight ratio of about 100:1 to about 1:1 or about 50:1 to about 1:1 or about 10:1 to about 1:1.

5. The sensing composition of claim 1, wherein the stabilizing agent is an albumin.

6. The sensing composition of claim 1, further comprising a pH buffer.

7. The sensing composition of claim 1, wherein the redox mediator comprises a polymer and an electron transfer agent.

8. The sensing composition of claim 7, wherein the polymer comprises poly(4-vinylpyridine), poly(1-vinylimidazole), poly(thiophene), poly(aniline), poly(pyrrole), or poly(acetylene).

9. The sensing composition of claim 7, wherein the polymer comprises a polymer or copolymer repeat unit comprising at least one pendant pyridinyl group, imidazolyl group, or both a pyridinyl and imidazolyl group.

10. The sensing composition of claim 7, wherein the electron transfer agent comprises a transition metal complex.

11. The sensing composition of claim 10, wherein the transition metal complex comprises osmium, ruthenium, iron, cobalt, vanadium, or a combination thereof.

12. The sensing composition of claim 11, wherein the transition metal complex is an osmium transition metal complex comprising one or more ligands, wherein at least one ligand comprises a nitrogen-containing heterocycle.

13. The sensing composition of claim 12, wherein the redox mediator comprises an osmium complex bonded to a poly(4-vinylpyridine)-based polymer.

14. The sensing composition of claim 1, further comprising a cross linking agent.

15. The sensing composition of claim 14, wherein the cross linking agent is a polyepoxide, cyanuric chloride, N-hydroxysuccinimide, an imidoester, epichlorohydrin, or a combination thereof.

16. The sensing composition of claim 15, wherein the cross linking agent is a polyethylene glycol diglycidylether (PEGDGE).

17. The sensing composition of claim 2, wherein the pyruvate oxidase is attached to the redox mediator.

18. A sensing electrode comprising a working electrode comprising a pyruvate sensing layer comprising the sensing composition of claim 1.

19. The sensing electrode of claim 18, wherein the pyruvate sensing layer is continuous.

20. The sensing electrode of claim 18, wherein the pyruvate sensing layer is discontinuous.

21. The sensing electrode of claim 18, further comprising a membrane overcoating at least the pyruvate sensing layer.

22. The sensing electrode of claim 21, wherein the membrane is permeable to pyruvate.

23. The sensing electrode of claim 22, wherein the membrane has reduced permeability to TPP relative to pyruvate.

24. The sensing electrode of claim 21, wherein the membrane comprises poly(4-vinylpyridine).

25. A system for sensing pyruvate, comprising
an electrode comprising the sensing composition of claim 1; and
a circuit configured for electrochemical detection of pyruvate at the electrode.

26. A system for sensing pyruvate comprising
a working electrode,
a sensing element disposed on at least a portion of the working electrode, and
a circuit configured to connect and disconnect with the working electrode,
wherein
the sensing element comprises the sensing composition of claim 1, and
the sensing element is configured to accumulate charge derived from pyruvate reacting with the pyruvate oxidase for a set period of time.

27. The system of claim 25, further comprising an implantable portion configured for inserting into a tissue, wherein the working electrode is disposed on the implantable portion.

28. A method for sensing pyruvate comprising:
contacting a sample comprising pyruvate with the sensing electrode of claim 18; and
detecting an accumulated charge derived from pyruvate reacting with the pyruvate oxidase and the redox mediator.

29. A method for sensing pyruvate comprising:
connecting the sensing electrode of claim 18 to a circuit to provide a steady state current;
disconnecting the sensing electrode from the circuit;
contacting the sensing electrode to an analyte comprising pyruvate;
accumulating charge derived from pyruvate reacting with the pyruvate oxidase and the redox mediator for a set period of time;
connecting the sensing electrode to a circuit after the set period of time; and
measuring a signal from the accumulated charge.

30. The method of claim 29, wherein the set period of time is 30 seconds or more.

31. The system of claim 26, further comprising an implantable portion configured for inserting into a tissue, wherein the working electrode is disposed on the implantable portion.
